(19) United States
(12) Patent Application Publication
(10) Pub. No.: US 2025/0352995 A1
(43) Pub. Date: Nov. 20, 2025

(54) SYSTEM AND METHODS FOR CONCURRENT GERMFREE AND GNOTOBIOTIC INFRASTRUCTURE

(71) Applicant: H. Lee Moffitt Cancer Center and Research Institute, Tampa, FL (US)

(72) Inventor: Robert Engelman, Tampa (FL)

(21) Appl. No.: 18/854,141

(22) PCT Filed: Apr. 5, 2023
(86) PCT No.: PCT/US2023/065387

§ 371 Date: Oct. 4, 2024

Related U.S. Application Data
(60) Provisional application No. 63/328,003, filed on Apr. 6, 2022.

(51) Int. Cl. B01L 1/02

(52) U.S. Cl. CPC B01L 1/025; B01L 2200/028

(57) ABSTRACT

Provided are systems and methods for performing germfree or concurrent germfree and gnotobiotic animal studies in the same room. The system can include one or more isolation individually ventilated cages equipped with HEPA-filtered positive-pressurization housing at least one animal that is germfree, a fecal transplantee, or gnotobiotic. An air handling unit provides filtered air to each of the IsoIVC-Ps when stored on a support rack. Each of the IsoIVCPs can be self-hermetically sealed when removed from the support rack. The system can also include one or more isolators, where each isolator is an aseptic glovebox isolator comprising a hydrogen peroxide vapor module. Concurrent animal studies can be performed by sterilizing, via autoclave, the IsoIVC-Ps, which are then inserted into the isolator. The animal-occupied IsoIVCPs are hermetically sealed while in the isolator and sterilized by hydrogen peroxide vapor. Then the animal-occupied IsoIVC-Ps can be opened inside the isolator for study.

This application claims the benefit of and priority to U.S. Provisional Application Ser. No. 63/328,003, having the title “SYSTEM AND METHODS FOR CONCURRENT GERMFREE AND GNOTOBIOTIC INFRASTRUCTURE”, filed on Apr. 6, 2022, the disclosure of which is incorporated herein by reference in its entirety.

### BACKGROUND

Mice (e.g., inbred, hybrid, congenic, genetically-engineered) are the model placental mammal most relied on for preclinical studies of the basic biology of human life, with thousands of isogenic mouse lines established to study the basic biology of human development, health, and disease, and for the preclinical modeling of novel therapeutics.

Increasingly human health, disease, and therapeutic outcomes are associated with influences of the microbiota (i.e., all of the microbes, including bacteria, viruses, fungi, and archaea that inhabit various body niches, including surfaces of the gastrointestinal tract, respiratory tract, and skin). Studies defining this mutualistic relationship between microbes inhabiting the human body, and the differentiated cells and tissues comprising human organ systems are hampered by insufficiently developed germfree (i.e., microbe free) and gnotobiotic (i.e., defined microflora) mouse model infrastructure technology.

Longitudinal studies of the mammalian microbiome require core capabilities that provide specialized supporting preclinical infrastructure, technical proficiencies, and instrumentation for the bioexclusion isolation housing and use of germfree and gnotobiotic mice, ensuring unaltered, uncontaminated microbial status of each model. To date, infrastructural systems for evaluating host-microbe interactions have relied on redundant rooms using either flexible film or rigid isolators, which are space inefficient, utilize hazardous liquid sterilants, expensive, difficult to handle, and problematic from an experimental access and design perspective, with germfree colonies and gnotobiotic study cohorts in separate rooms and in separate isolators. These needs and other needs are satisfied by the present disclosure.

### SUMMARY

Embodiments of the present disclosure provide systems for performing germfree or concurrent germfree and gnotobiotic animal studies in the same room, methods of use, and the like.

An embodiment of the present disclosure includes a system for performing concurrent animal studies in the same room. The system can include one or more isolation individually ventilated cages equipped with HEPA-filtered positive-pressurization (IsoIVC-P), wherein each of the cages houses at least one animal that is germfree, a fecal transplantee, or gnotobiotic. The system can further include one or more support racks comprising an air handling unit, wherein the air handling unit provides filtered air to each of the IsoIVC-Ps when stored on the support rack. Each of the IsoIVC-Ps can be self-hermetically sealed when removed from the support rack. The system can also include one or more isolators, where each isolator is an aseptic glovebox isolator comprising a hydrogen peroxide vapor module.

An embodiment of the present disclosure also includes methods for performing concurrent animal studies in the same room. The method includes sterilizing, via autoclave, one or more isolation individually ventilated cages with HEPA-filtered positive-pressurization (IsoIVC-P). The method further includes providing an animal to each of the one or more sterilized IsoIVC-Ps. The animal-occupied IsoIVC-Ps are then inserted into an isolator. The isolator can be an aseptic glovebox isolator equipped with a hydrogen peroxide vapor module. The animal-occupied IsoIVC-Ps are hermetically sealed while in the isolator. Then, the animal-occupied IsoIVC-Ps are sterilized by hydrogen peroxide vapor. Then the animal-occupied IsoIVC-Ps can be opened inside the isolator to perform a study.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure.

FIGS. 1A-1H are camera images of equipment used in accordance with embodiments of the present disclosure. FIG. 1A shows a Bioquell Proteq model room hydrogen peroxide vaporizer. FIGS. 1B-1E show hydrogen peroxide vapor monitoring inside and outside of an IsoIVC-P. FIG. 1F shows external sterilization of a Taconic shipper. FIG. 1G shows Geobacillus stearothermophilus spores sealed inside pouches that were incubated in trypticase soy broth at 60° C. for 7 days. FIG. 1H shows biological indicators used to detect HPV levels.

FIGS. 2A-2T are camera images of preliminary QUBE studies without mice, assessing whether a QUBE HPV cycle can decontaminate the exterior surfaces of an IsoIVC-P and Taconic germfree shipper while leaving the interiors of both undisturbed. FIG. 2M shows the growth of biological indicators that had been placed in the IsoIVC-P interior during HPV sterilization of its exterior, showing that interiors remained undisturbed. FIGS. 2N and 2O show the IsoIVC-Ps complete with bedding and enrichment items on storage racks and in the isolators, respectively. FIG. 2P shows technicians working with IsoIVC-Ps in the isolator. FIGS. 2Q and 2R show that no HPV, and only negligible changes to ambient temperature, humidity, and pressure registered inside the sealed IsoIVC-P during a HPV sterilizing cycle. FIG. 2S shows that a HPV low-level sensor enclosed inside the sealed IsoIVC-P during the QUBE's sterilization cycle, when removed after cycle completion, detected no remnant HPV inside the QUBE chamber. FIG. 2T shows the IsoIVC-P racking system (left), and room HPV sterilizing generator (center) and the QUBE (right).

FIGS. 3A-3E provide embodiments of an anesthesia system for use in the systems described herein. FIG. 3A is a schematic representation, illustrating that oxygen-bearing isoflurane as an inhalant general anesthetic enters the QUBE via a dummy plug connection that passes through the QUBE chamber floor, through a tri-clover port, through a tri-clover adapter fashioned with a two-sided barbed connector, through the Pall filters, and is delivered to the mouse in either an induction chamber or placed at a rodent-specific anesthetic facemask. FIG. 3B shows the two-sided barbed connector. FIGS. 3C and 3D show the tri-clover port. FIG. 3E shows the tri-clover adapter (arrow), the two Pall filters, the induction box, the facemask, and the connecting tubing.

FIGS. 4A-4E are camera images according to embodiments of the present disclosure. FIG. 4A shows a rack of IsoIVC-P in use, occupied by germfree colony mice. FIG. 4B shows the process of loading the QUBE with IsoIVC-P and all supplies and instruments needed for mouse use inside the QUBE. FIG. 4C shows supplies and an IsoIVC-P being HPV sterilized during a loaded cycle. FIG. 4D shows the QUBE after a sterilizing loaded cycle during processing mode when IsoIVC-P are opened, and mice are used and handled. FIG. 4E shows both the IsoIVC-P racking system (left) and the QUBE in use (right).

FIGS. 5A-5C are graphs demonstrating the conditions inside and outside of the IsoIVC-P during a QHPV loaded cycle, indicating an IsoIVC-P exterior sterilizing effect by HPV, while the IsoIVC-P interior experienced only a modest increase in ambient temperature in accordance with embodiments of the present disclosure. FIG. 5D is a camera image of a handheld low-level H₂O₂ monitor that was used to confirm that HPV levels had degraded to <1 ppm inside the QHPV.

FIGS. 6A-6C are camera images illustrating methods according to the present disclosure. During twelve QHPV processing modes, airborne live microbes were recovered using culture settle plates (FIG. 6A, arrow) that had been placed in an active air sampler (FIGS. 6A, 6B, blue arrow), and total airborne particles of two size thresholds were counted using a light-scatter airborne particle counter (FIG. 6C, black arrow). Average airborne particle counts of twelve QHPV processing modes (FIG. 6D) remained low while animals were handled and gavage administered, well below requirements of ISO 14644-1 Class 5. FIG. 6E shows readcounts of microbiome shotgun sequencing of fecal pellets of representative mice after 6 weeks of bi-weekly gavage of either sterile PBS (from left, lane 1), two unique SPF FMT (lanes 6 & 7), or ASF FMT (lane 5), and of germfree colony mice of either the BALB/c (lane 2), C57BL/6 (lane 3), or Swiss Webster (lane 4) murine lines housed on the same IsoIVC-P rack.

FIG. 7 shows the relative abundance of the fecal bacterial microbiota of mice gavage administered FMT of one of three distinct flora, either an Altered Schaedler's Flora (ASF) slurry, or one of two SPF slurries derived from SPF murine colonies housed in separate vivarium, which remained distinct through the end of the six-week study.

FIG. 8 shows readcounts of microbiota shotgun sequencing of fecal pellets of 16 representative mice, 8 male mice (M1-6, 15-16) and 8 female mice (F38-41, 28, 29, 31, 35), prior to gavage, and after 1 week, 3 weeks, and 5 weeks of weekly gavage of either PBS or SPF FMT. A mono-contaminant, Paenibacillus macerans in a single female mouse (week 0, F41) spread to members of the same PBS gavage female cohort handled during the same processing mode (weeks 1-5, F38-41), but not to members of the germfree male cohort also receiving PBS by gavage during separate processing modes (M1-4), or to germfree colony mice housed nearby on the same rack and processed separately (data not shown).

FIG. 9 shows the percentage of murine colony fecal specimens negative by PCR for bacterial 16s rRNA and microbial cultures during the study (green circles). Number of germfree IsoIVC-P (blue diamonds) and number of FMT and ASF IsoIVC-P (red triangles) housed during the study. Dates of germfree studies are indicated by blue bars and dates of gnotobiotic studies are indicated by red bars.

FIGS. 10A-10D are camera images showing the infrastructure used for germfree murine colony production (FIG. 10A), for tumor growth studies (FIG. 10B), for microsurgical procedures (FIG. 10C), and for germfree isoflurane inhalational anesthesia procedures that incorporated in-line anesthesia gas filters (FIG. 10D, white arrow).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biology, immunology, and the like, which are within the skill of the art.

The following examples are put forth so as to provide those of ordinary skill in the art with a complete disclosure and description of how to perform the methods and use the systems and methods disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent. law and the term is open-ended, allowing for the presence of more than that which is recited so long as basic or novel characteristics of that which is recited is not changed by the presence of more than that which is recited, but excludes prior art embodiments.

### Definitions

Isobiotic, as used herein, refers to animals with an identical stable microbiota composition.

Isogenic, as used herein, refers to a population with essentially identical genes.

Germ-free organisms, as used herein, refer to multi-cellular organisms that are devoid of internal and external microorganisms as determined by convention “within the limitations of the detection methods available.

Gnotobiotic, as used herein refers to a multicellular organism or an environment for maintaining a multicellular organism in which all microorganisms are defined and known or excluded.

### Abbreviations

Isolation individually ventilated cage with HEPA-filtered positive-pressurization (IsoIVC-P); individually-ventilated cage (IVC); hydrogen peroxide vapor (HPV).

### General Discussion

In accordance with the purpose(s) of the present disclosure, as embodied and broadly described herein, embodiments of the present disclosure, in some aspects, relate to systems and methods for animal studies and care.

In general, embodiments of the present disclosure provide for methods and systems for performing concurrent germfree and/or gnotobiotic mouse studies in the same room, including isolators, hydrogen peroxide vapor sterilization, and isolation individually ventilated cages with HEPA-filtered positive-pressurization.

The present disclosure includes a method for performing concurrent multiple murine strain germfree colony production and germfree and/or gnotobiotic mouse experimental cohort studies in the same room. The method can include mice on the same housing rack, through a single isolator, where the method includes sterilizing, via autoclave, the interior of one or more isolation individually ventilated cages with HEPA-filtered positive-pressurization (IsoIVC-P), and sterilizing its exterior, prior to its opening, by hydrogen peroxide vaporization. The method further includes providing an animal to each of the one or more sterilized IsoIVC-Ps to form one or more animal-occupied IsoIVC-Ps, then providing the one or more animal-occupied IsoIVC-Ps to the isolator, where the isolator is an aseptic glovebox isolator with integral hydrogen peroxide vaporizer, and wherein the one or more animal-occupied IsoIVC-Ps are hermetically sealed while in the isolator undergoing exterior sterilization. The one or more animal-occupied IsoIVC-Ps can be sterilized via hydrogen peroxide vapor and then opened to assess while in the isolator, thereby contributing to the performance of a study, or the production of a colony. Advantageously, animals from differing germfree or gnotobiotic cohorts, each with unique microbial consortia, can be housed in their IsoIVC-Ps adjacent to one another on the same housing rack without contamination, and entered separately into the isolator with integral hydrogen peroxide vaporizer for use. Studies and procedures can then be performed in the isolator on mice that have like microbial consortia.

Embodiments of the present disclosure include a system for performing concurrent germfree colony production and/or germfree and gnotobiotic mouse experimental studies in the same room as above, including one or more isolation individually ventilated cages with HEPA-filtered positive-pressurization (IsoIVC-P); one or more support racks comprising an air handling unit, wherein the air handling unit provides filtered air to each of the IsoIVC-Ps when the one or more IsoIVC-Ps are stored on the support rack, wherein each of the one or more IsoIVC-Ps is self-hermetically sealed when removed from the support rack; and one or more isolators, where each isolator is an aseptic glovebox isolator comprising an integral hydrogen peroxide vaporizer.

Previous studies have shown that it is possible to maintain germfree mice in a bioexclusion, hermetically-sealed, isolation individually ventilated cage with HEPA-filtered positive-pressurization (IsoIVC-P) for 3-12 weeks (1, 2), that mice with an infectious agent in one individually-ventilated cage (IVC) do not contaminate uninfected mice in a neighboring IVC on the same rack (3), and that IVC housing does not increase inter-cage (4), or inter-individual variation of the microbiota (5), but may influence temporal dynamics of complex microbiota, with some relative abundances clustered in mice housed in either isolators or IVC, longitudinally (6).

However, the current germfree & gnotobiotic mouse model equipment and methods have numerous disadvantages. They can be space inefficient, requiring: redundant isolators, often in separate rooms, for each germfree mouse line in colony production; separate isolators for each gnotobiotic mouse study; and separate surgical/procedural isolators for implementing research aims. The present methods can be hazardous to staff, utilizing toxic nebulized sterilants (e.g., sodium chlorite) to decontaminate isolators, transfer sleeves and ports, or liquid sterilants (e.g., peracetic acid) in dunk tanks to decontaminate hermetically-sealed cages that are then opened in a biosafety cabinet. The cabinet can be laminar flow ventilated but have an open sash, introducing risk of contamination. The methods can have slow throughput and be logistically difficult, requiring extensive preparation, docking, and the use of large metal transfer cylinders for autoclave sterilization of supplies needed inside isolators, and the disposal and rinsing each day of liquid sterilants used in dunk tanks. The methods can also present high risk, since germfree colony housing is currently reliant on high efficiency particulate arresting (HEPA) filtered air insufflated flexible-film isolators, or rigid glovebox isolators, enclosing open cages of germfree mice, and contamination results in loss of the isolator's entire contents.

The systems and methods described herein provide a robust system for investigating host-microbe interactions in germfree & gnotobiotic mouse models and provide numerous advantages over existing systems and methods. Advantageously, they are high-throughput such that multiple germfree mouse lines and gnotobiotic mouse experimental cohorts comprised of various microbial consortia can all be housed concurrently in the same room, on the same rack, and used in the same room, separately opened in the same isolator. The described systems and methods are highly space efficient with redundancy of germfree colonies established at the primary (e.g. IsoIVC-P+QUBE) rather than secondary enclosure level (e.g. flexible-film or rigid isolator enclosing open top cages). The methods and systems are also accessible, safe, and streamlined, thereby accelerating germfree and gnotobiotic infrastructure that facilitates rapid scientific advancement. The environments provided by the described systems and methods are rigorous and controlled for reproducible studies using germfree and gnotobiotic mice. Additionally, the systems and methods described herein are low risk, since contamination results in the loss of only the affected primary enclosure. IsoIVC-P and germfree colonies are represented by numerous redundancies of each IsoIVC-P.

Advantageously, the systems and methods as described herein are highly space efficient and can allow for multiple germfree murine colonies and germfree or gnotobiotic mice experimental cohorts each with unique microbial consortia on one supporting rack, in one room without cross-contamination, allowing for concurrent germfree and gnotobiotic mouse studies using the same glovebox isolator with integral hydrogen peroxide vaporizer for all husbandry and procedural uses.

The system and methods described herein integrate two existing technologies from different fields of endeavor. This integrated system of equipment and procedures accomplishes functions that neither single component alone can provide. The system integrates at least one isolator having a hydrogen peroxide vapor (HPV) decontaminating cycle and at least one isolation cage, wherein the isolation cage can include HEPA filtered positive pressurization.

Advantageously, the methods described herein can maintain germfree colony mice long-term, and ensures that ASF defined flora mice and SPF FMT administered experimental mice do not contaminate germfree mice in neighboring IsoIVC-P. Even following a contamination event, the described combination of technologies allows research to continue in the same space, with no downtime due to decontamination and follow-up testing of equipment and did not necessitate depopulation of valuable research animals.

Further, the infrastructure does not increase the variability of FMT complex microbiota, has lower risk and fewer hazards than traditional infrastructure, with no liquid sterilant required. It is space efficient and versatile, permitting germfree husbandry, inhalational anesthesia, and microsurgical procedures all in the same footprint. It is highly reliable and more accessible, increasing the potential for new discoveries of the microbiome.

Although the germfree and gnotobiotic models referred to herein are mouse models/mouse colonies, other host models can be used depending on the needs of the experiment, as can be envisioned by one of ordinary skill in the art.

### Isolator

In some embodiments, the isolator is a glovebox isolator designed for clean-room manufacture of sterile products (e.g., pharmaceutical manufacturing & compounding, gene & cell therapy) with an integrated 35% hydrogen peroxide vaporizer that establishes a European Grade A/ISO 5 and United States Federal Standard 209E/class 100/ISO 5 aseptic work environment via a hydrogen peroxide vapor (HPV) decontaminating cycle and HEPA-filtered unidirectional positive pressurization. In some embodiments the isolator is a Bioquell QUBE (Bioquell, Inc., Horsham, PA).

The isolator, also referred to as an aseptic processing workstation, comprises a chamber wherein pressure in the chamber and airflow can be controlled in when fresh air is included in the airflow and when in re-circulatory mode. Recirculatory mode includes when running a decontamination cycle to contain the sterilant, during distribution (gassing phases), and during removal of sterilant.

The isolator includes apparatuses such that the chamber can be sterilized by a sterilant vapor such as hydrogen peroxide vapor. The relative humidity in the enclosure is reduced and a carrier gas circulated. A sterilant vapor is provided to the circulating carrier gas sufficient to saturate substantially the gas whereby on cooling in the enclosure, a condensate of the sterilant vapor is formed on surfaces in the enclosure for a predetermined period of time and the condensate extracted from the enclosure.

### Isolation Cage

In some embodiments, the isolation cage is a bioexclusion, hermetically-sealed, isolation cage individually ventilated with HEPA-filtered positive-pressurization (IsoIVC-P). In some embodiments, the isolation cage is a Tecniplast IsoIVC-P (Tecniplast, Buguggiate, Italy). On its supporting rack, IsoIVC-P are supplied with HEPA-filtered air delivered via an air supply plenum and vertical manifolds from an adjoining air handling unit (AHU) equipped with redundant blowers and battery backup. Each IsoIVC-P enclosure also has a cage-level HEPA filter positioned in the air supply stream, and when removed from its supporting rack, IsoIVC-P cages have self-closing air nozzles so that each remains hermetically sealed and positive-pressurized.

Advantageously, the methods and systems can be scaled by using multiple isolators and multiple racks of cages.

Existing systems house animals from a single germfree or gnotobiotic population in open top cages enclosed together in a flexible-film or bubble isolator, requiring a greater laboratory footprint. Existing systems and methods used for germfree and gnotobiotic mice (1-8) do not utilize hydrogen peroxide vapor, do not utilize a glovebox isolator with an integral hydrogen peroxide vaporizer, and do not provide an accessible, high throughput infrastructure for germfree colony production with multiple levels of redundancy and studies involving germfree and gnotobiotic mice.

In other embodiments, the systems and methods described herein could be used for up to level four biocontainments by providing HEPA filtration on the exhaust rather than the intake of the isolator.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

Preliminary IsoIVC-P HPV Safety & Efficacy Tests: The QUBE glovebox isolator decontaminates by conversion of liquid, concentrated 35% hydrogen peroxide into a gaseous phase by flash vaporization, resulting in HPV, which is rapidly, broadly antimicrobial and sporicidal through oxidation, dispersed onto all exposed surfaces sealed within the QUBE chamber (9-11). (Prior to QUBE purchase, preliminary tests were conducted using an owned Bioquell Proteq model room hydrogen peroxide vaporizer, an IsoIVC-P, and a Taconic shipper.

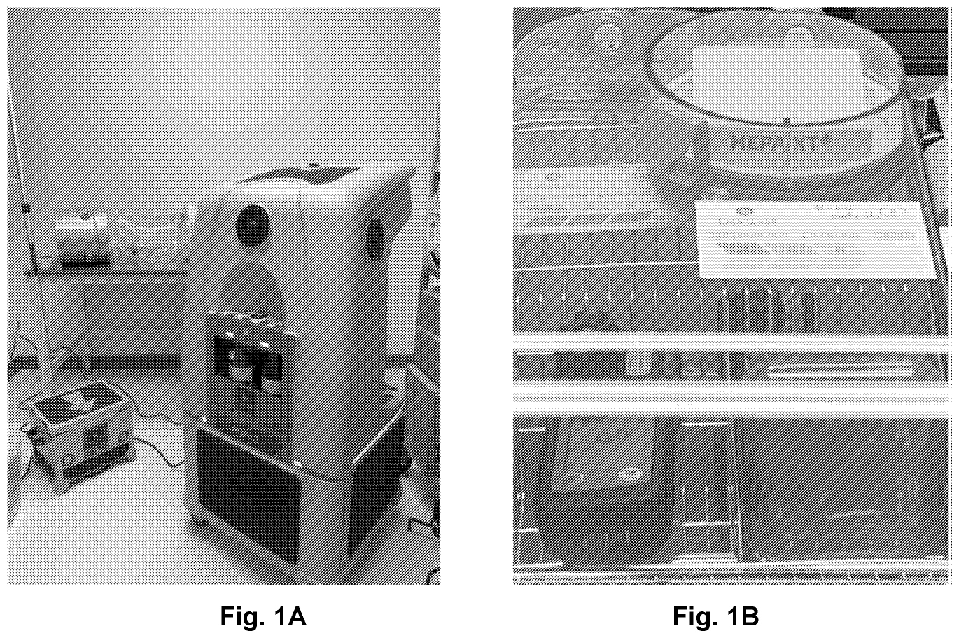

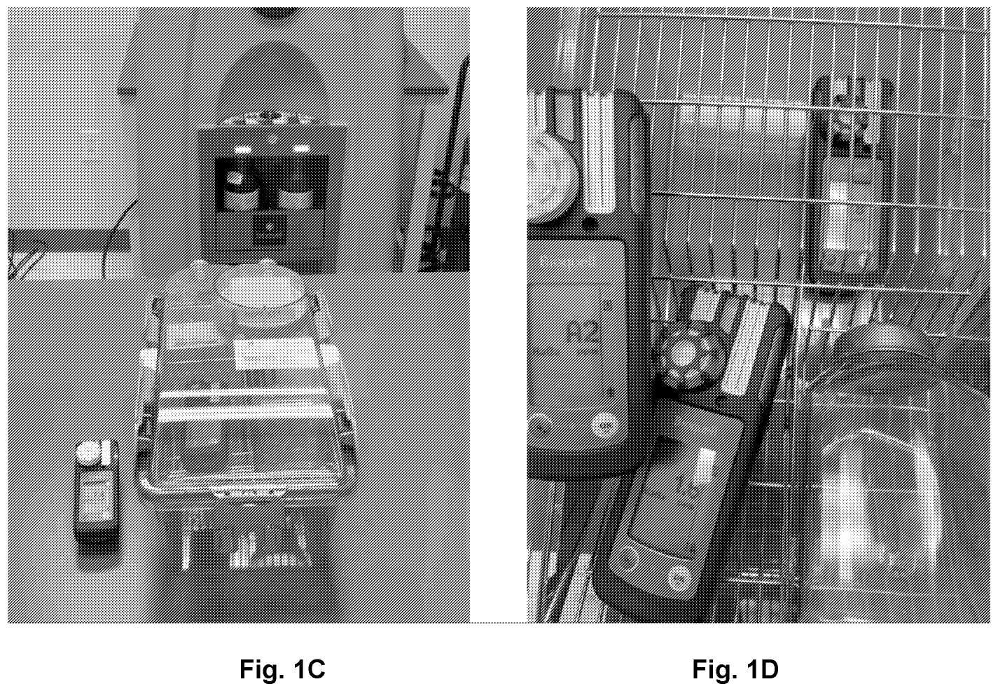

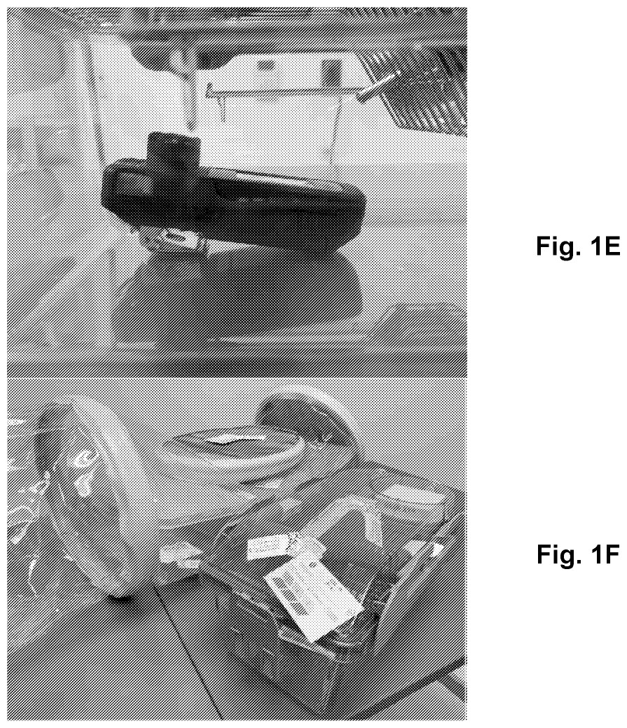

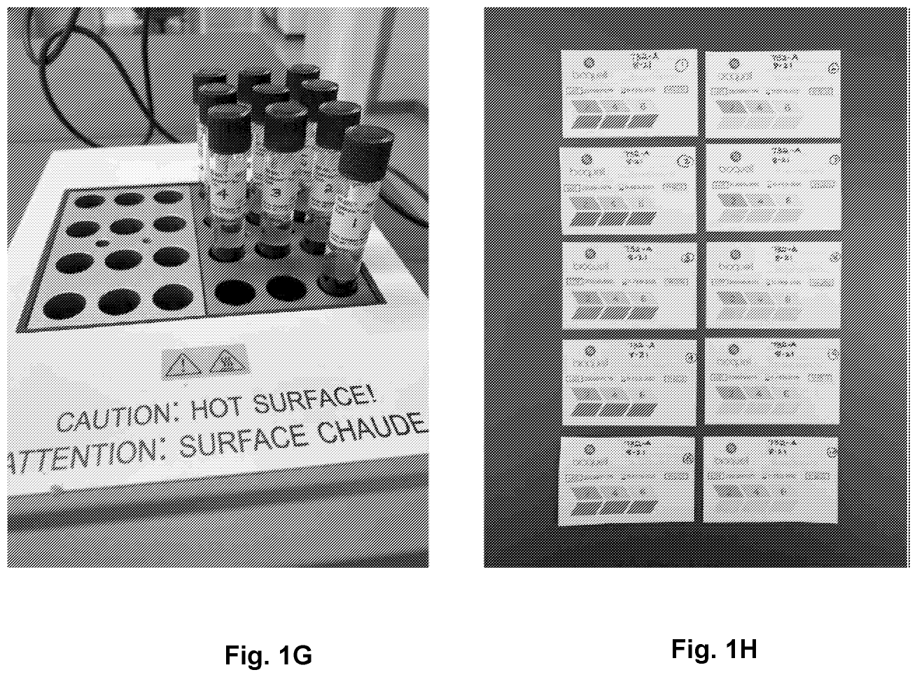

Biological indicators (Bioquell) were used to detect HPV levels capable of a 6-log reduction in microorganismal growth from Geobacillus stearothermophilus spores sealed inside pouches that were incubated in trypticase soy broth at 60° C. for 7 days (FIG. 1G).

Chemical indicators (Bioquell) provided a semiquantitative visual indication of 2-log, 4-log, 6-log, or greater than 6-log microbial deactivating levels of HPV (FIG. 1H).

After each HPV decontaminating cycle completion, HPV degrades to water and oxygen to undetectable <1 ppm levels, which were also confirmed using a handheld hydrogen peroxide monitor (Drager Safety, Lubeck, Germany). Chemical and biological indicators were placed inside and outside the sealed Iso-IVC-P and Taconic shipper to document safe exterior antimicrobial decontamination with no HPV detected in IsoIVC-P or Taconic shipper interiors (FIGS. 1A-1F).

Preliminary QUBE Safety & Efficacy experiments were conducted first without mice, and then with specific pathogen free (SPF) mice.

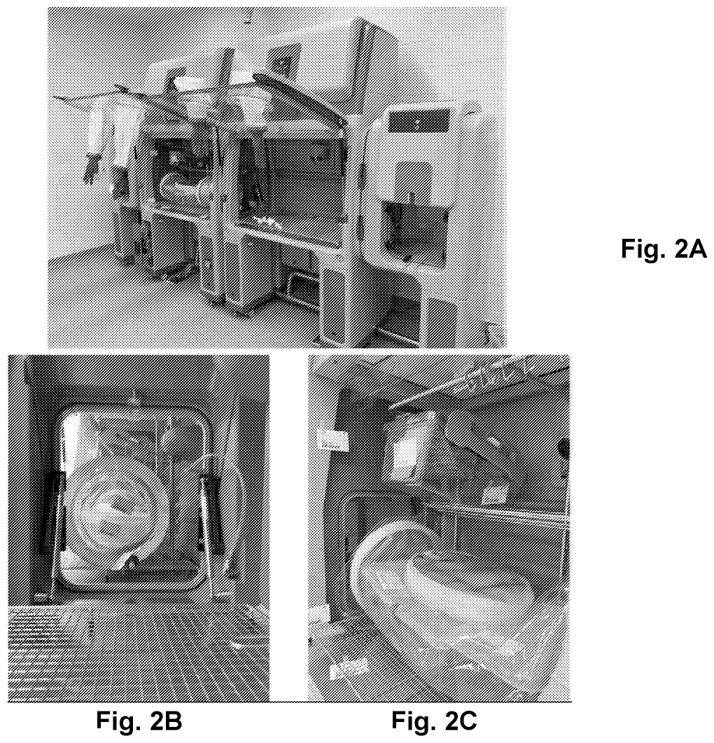

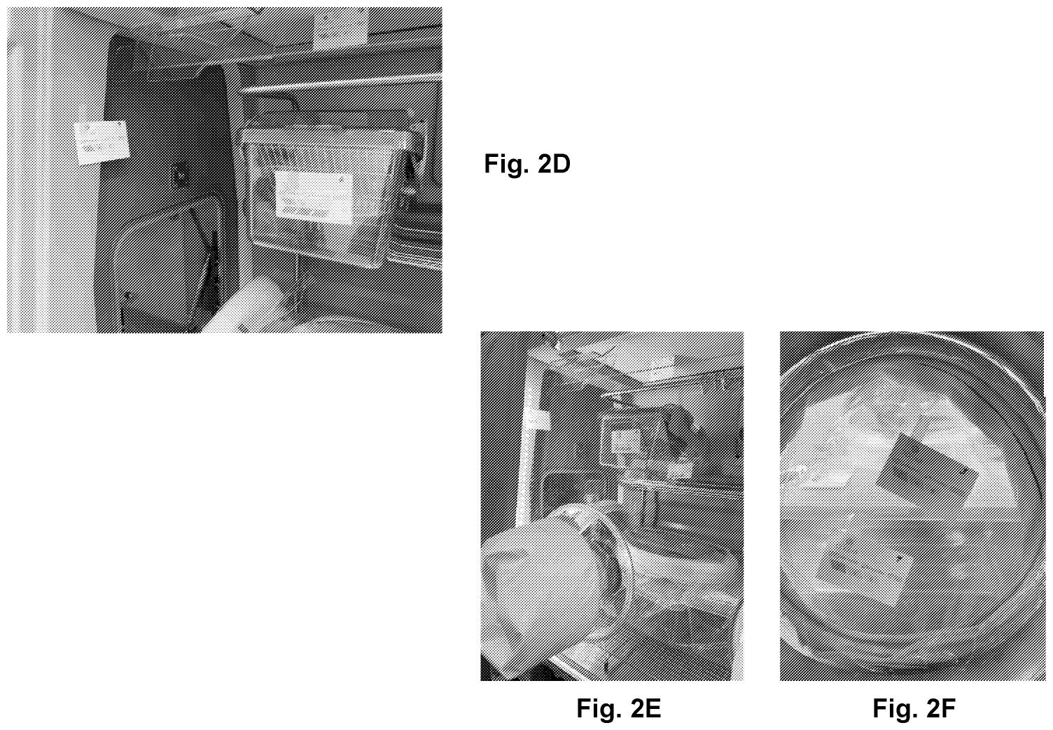

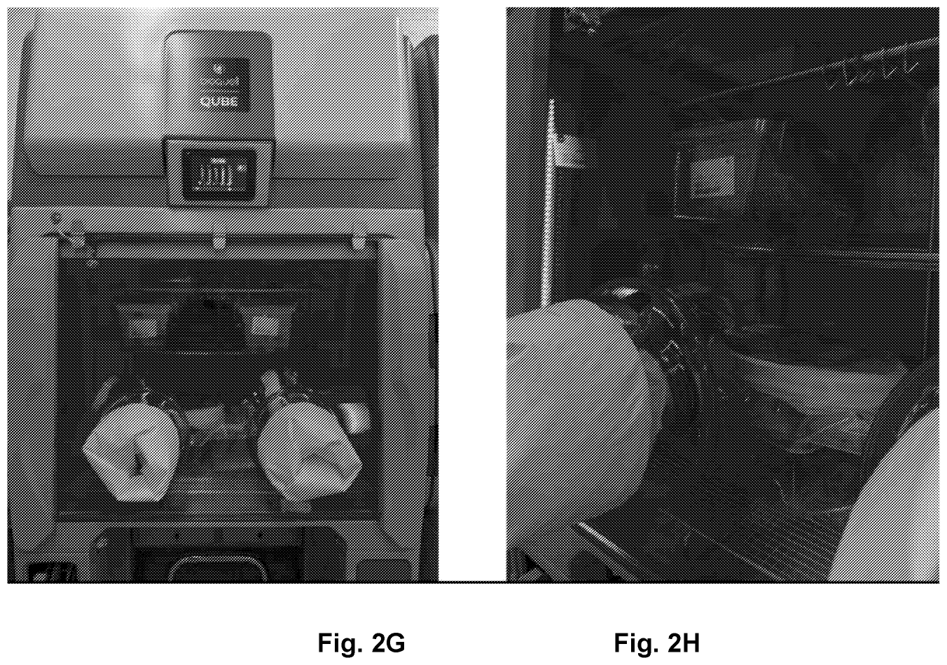

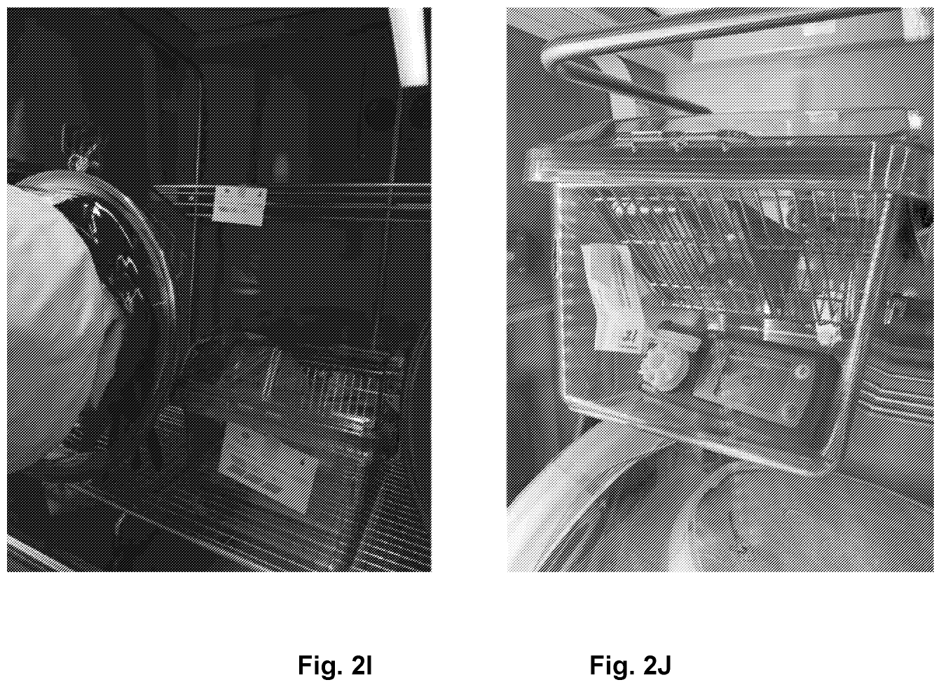

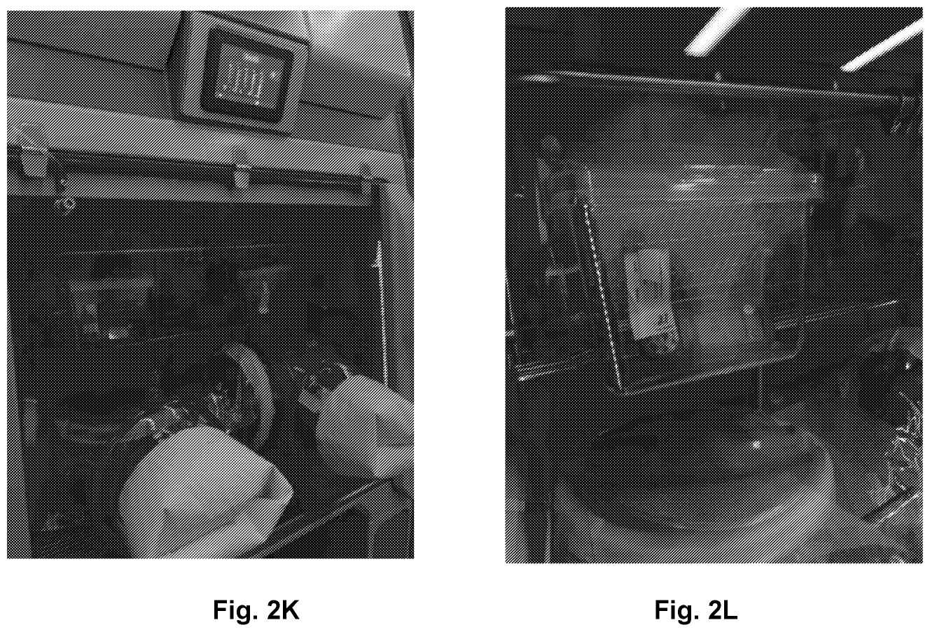

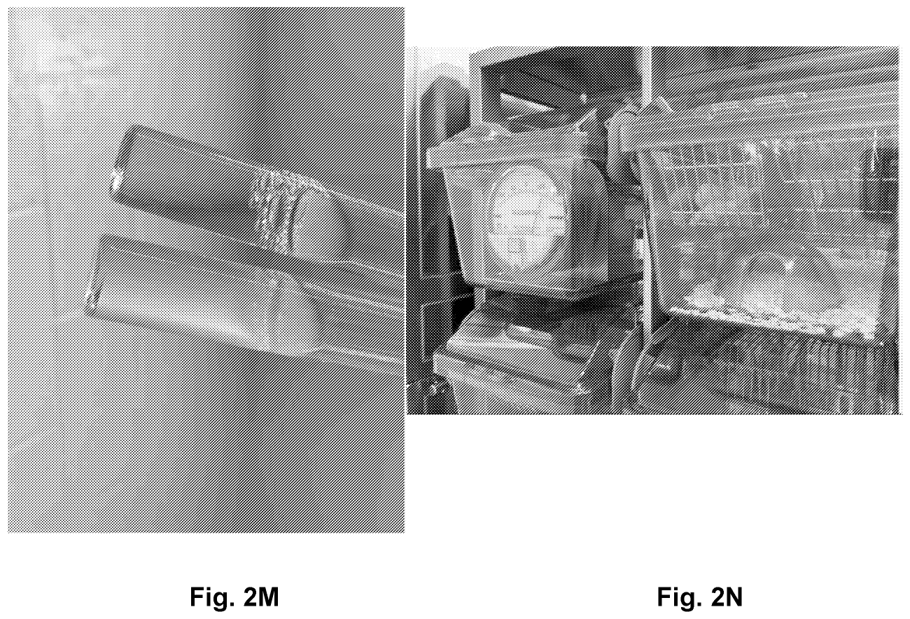

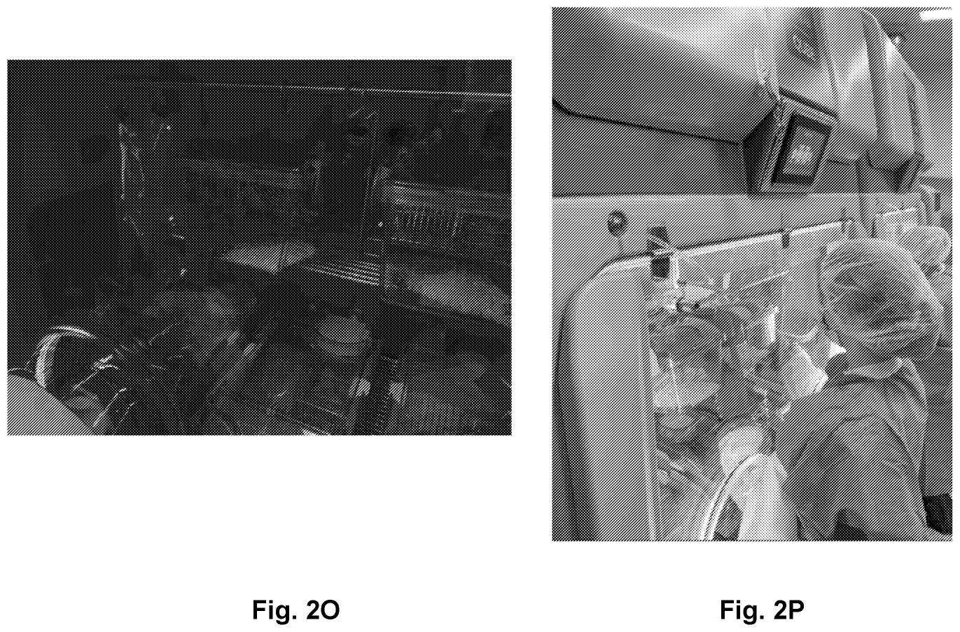

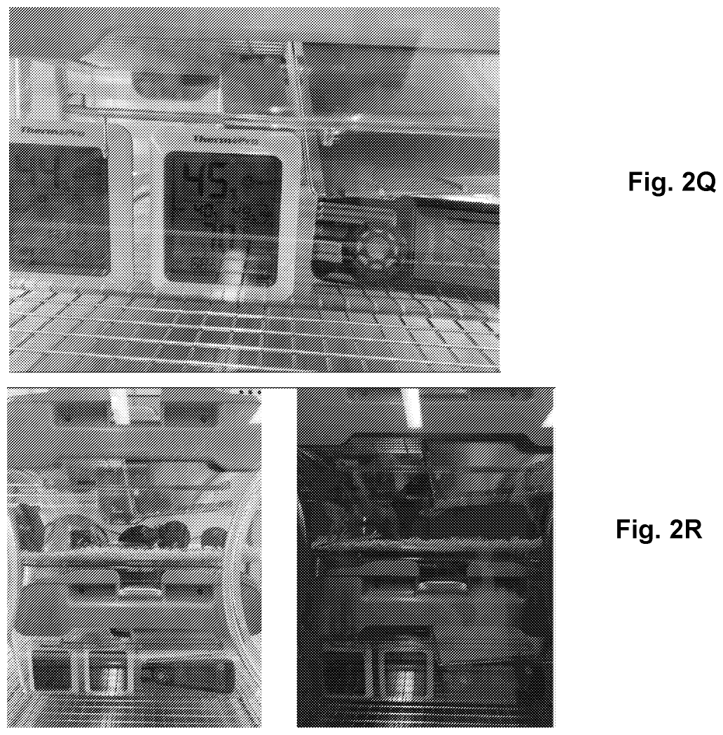

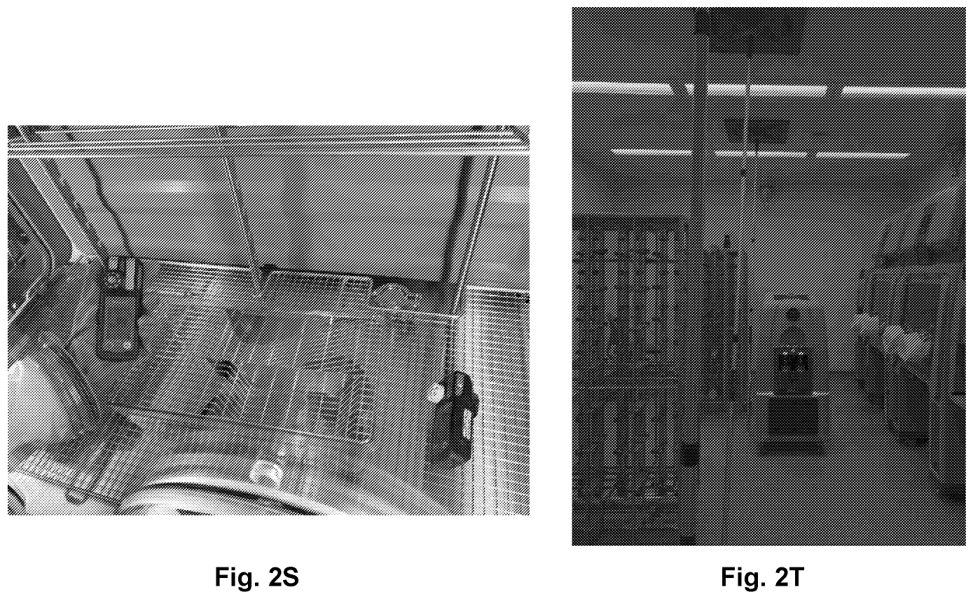

Preliminary QUBE studies without mice assessed whether a QUBE HPV cycle can decontaminate the exterior surfaces of an IsoIVC-P and Taconic germfree shipper, (shown in FIGS. 2A-2T). Taconic shipper (Taconic Biosciences, Germantown, NY) is designed for the freight delivery of germfree mice, comprised of a large, flexible vinyl sleeve (12″ diameter ×3½″ long, with two filtered openings) accommodating three cages for mice, each 11½″×7½″×5″. Tests documented that while interior HPV levels inside the IsoIVC-P and Taconic shipper remained undetectable <1 ppm, exterior indicators documented >6-log decontamination.

Chemical and biological indicators were placed inside and outside the sealed Iso-IVC-P and Taconic shipper. In addition, swab specimens of the QUBE HPV decontaminated exterior surfaces of the Taconic shipper and IsoIVC-P were collected and subjected to 16srRNA PCR amplification and aerobic, anaerobic, and fungal culture, with no amplicons and no growth observed.

Further, during a QUBE QHPV loaded cycle, decontaminating levels reach >1000 ppm HPV inside the QHPV chamber, while HPV levels in the sealed Iso-IVC-P remained <1 ppm, and temperature and humidity inside the sealed Iso-IVC-P were negligibly affected with interior temperatures between 28°-32° C. and interior relative humidity between 42-46%. In addition, QUBE interior pressurizations during processing are <75 Pa in the QHPV chamber (i.e., atmospheric pressure at sea level is 101,325 Pa or 14.7 PSI) with no effect to animal welfare. Conclusion: Preliminary QUBE studies without and with SPF mice documented its safety and efficacy for germfree mouse use.

Preliminary IsoIVC-P Studies with mice (i.e., 5 C57BL/6, adult, 30 gm, male mice) were conducted to ensure animal welfare was supported for longer than a QUBE loaded decontaminating cycle.

Tecniplast advises that <5 minutes inside the hermetically-sealed IsoIVC-P is the maximum static limit supportive of mice.

However, IsoIVC-P interior volume is greater than 15 cm×30 cm×15 cm=6,750 cm3, providing >6,700 ml of air, in which there are 1,407 ml of O₂ (21%). Murine O₂ consumption is approximately 60-80 ml/kg/minute, so 5 mice×30 gm/mouse=150 gm, or 80 ml/kg×0.150 kg=12 ml/minute consumed by 5 adult mice. Since, 1,407 ml O₂ are available in IsoIVC-P, and 12 ml O₂/min are consumed by 5 adult mice=117 minutes, or 1.9 hours, or approximately 1 hour and 54 minutes of O₂ should be available.

Under continuous direct observation, five (5) adult 30 gm male C57BL/6 mice remained very active for >90 minutes inside a hermetically-sealed IsoIVC-P (i.e., 2×QUBE HPV loaded cycle time of 45 minutes) at which time monitoring was ended, mice all still very active.

Preliminary IsoIVC-P studies with mice demonstrated that the hermetically sealed IsoIVC-P primary enclosure retained sufficient oxygen to support mice for >90 minutes, 2×QUBE HPV loaded cycle.

The QHPV loaded cycle is quick, <45 minutes, with sterilant dissipated by an integral aeration catalytic accelerator, ensuring HPV levels at cycle completion are below the long-term exposure limit 8-hour time-weighted average of <1 ppm inside the QHPV chamber prior to opening the Iso-IVC-P.

Preliminary Autoclave Sterilization Validation Initial validation experiments to confirm redundant autoclave readiness of programmed germfree sterilization cycles, as follows, Germ-free (GF) Iso-IVC cycle: 2.45 psia prevacuum, 121 C, 20 minutes, 2.45 psia drying vacuum. Germ-free (GF) liquid cycle: 122 C, 90 minutes, 2.45 psia vacuum (no pre- or post-pulse stages).

Bulk or Model 1000 autoclave were used for sterilizing interiors of IsoIVC-P with feed & bedding, or separately, Corning reverse osmosis (RO) animal drinking water reservoir bottles.

Steris Verify Steam Test Packs with Self Contained Biological Indicators (SCBI) and with Steris Verify integrator strips were placed in IsoIVC-P, deep in feed, deep in bedding, or inside empty cage bottle. After sterilization, all Verify integrator strips indicated effective sterilization in all IsoIVC-P, and all SCBI after incubation were “blue-purple” indicating no growth & effective sterilization.

Liquid germfree cycle was validated using Mag-Amp biological indicators suspended in RO drinking water reservoir bottles. After sterilization, no growth was recorded inside any Mag-Amp (i.e., turning growth medium from purple to yellow indicating a non-sterile “positive” test). The extended sterilization cycle did begin to “caramelize” the pH indicator color within the growth media, and media did appear “altered” post-sterilization, but it has been documented by Steris that caramelized media retains its ability to promote growth.

IsoIVC-P are assembled with bedding (i.e., autoclaved, irradiated, paper cellulose bedding, Shepard's Alpha Dry-IRR) and feed (i.e., autoclaved, irradiated, extruded chow, 5VOF-IRR 25 #, Irradiated Purina Select Rodent Lab Diet 50 IF/6F Auto) and interiors sterilized using autoclave steam sterilization.

IsoIVC-P are sterilized using a germfree autoclave cycle of 2.45 psia pre-vacuum, 121° C., 20 minutes, 2.45 psia drying vacuum while IsoIVC-P are unlatched, with white safety buttons up, with each assembled IsoIVC-P held tightly in place by the spring-loaded runners of the transport rack, with each IsoIVC-P supply air valve actuated open to permit steam to enter each interior. Cage-level HEPA filters are discarded after 10 sterilization cycles.

Autoclaved reverse osmosis (RO) drinking water is prepared in reservoir bottles using a germfree liquid sterilization cycle of 122° C., 90 minutes, 2.45 psia vacuum (i.e., no pre- or post-pulse stages). Baskets of reservoir bottles (i.e., Corning 1 L glass bottles) are filled with RO drinking water, capped loosely with red high temperature-resistant caps, and Tyek covered.

Bioquell Qube is an aseptic workstation, a rigid, glove-box isolator with an integral HPV decontamination system, which is loaded via its front access window. The Qube system has 2 large chambers, a hydrogen peroxide vapor (QHPV) module, and an extension (QEXT) module. Each of these 2 larger chambers is flanked by a smaller, material transfer device (QMTD) used for exiting processed material. The complete Qube system is controlled via a control panel on the QHPV module. The QHPV is used for decontaminating a load.

During decontamination mode, the QHPV provides assurance of a 6-log microbial reduction of the load placed inside the QHPV module. Two decontamination cycles are possible. Decontamination can be of a load placed inside the QHPV (i.e., QHPV loaded cycle), or of the empty Qube system with all interior doors open (i.e., System empty cycle).

During “use” or processing mode (after HPV decontamination), the Qube system provides a unidirectional airflow working zone that meets the classification requirements of ISO 14644-1 Class 5 (equivalent to EU cGMP Grade A and US FED STD 209E Class 100). During “use” or processing mode, the Qube system maintains an airflow rate of 573 m3/h and chamber pressure set points of 75 Pa for the QHPV, 50 Pa for the QEXT, and 36 Pa for each of the QMTDs (i.e., a positive pressure gradient from the QHPV to the room).

Both the QHPV and QEXT have side doors fitted with pneumatic inflatable seals, which automatically inflate when the door is manually closed. All doors are interlocked, ensuring that doors cannot be opened during decontamination mode.

Each side door has an indicator light which is either lit continuous green when it is enabled and the door can be opened, not illuminated when it is disabled and cannot be opened, flashing red when there is an alarm involving the door seal, or flashing green when an action is required of the door (e.g., “open the door” prior to the start of a System empty cycle, or “close the door” at the end of a System empty cycle).

Only one door of the QMTD module can be opened at a time. After opening the QMTD external door, there is a 2-minute clean-up time before the internal door of the same QMTD can be opened again.

### Challenges Overcome & Demonstrated by the Systems and Methods Described Herein

That germfree mice can be maintained long-term outside of an isolator had not been demonstrated for >12 weeks (1, 2) prior to the studies described herein.

That germfree mice can be maintained long-term concurrently in the same room on the same rack with multiple gnotobiotic studies without contamination had not been demonstrated prior to the studies described herein.

That HPV can be used with mice had not been demonstrated prior to the studies described herein.

That HPV can be used with IsoIVC-P, or IsoIVC-P can be used with HPV had not been demonstrated prior to the studies described herein.

That HPV can be used to ensure the consistent microbial quality of germfree and gnotobiotic inventories housed simultaneously in the same room on the same rack had not been demonstrated prior to the studies described herein.

That IsoIVC-P and the QUBE can be used together to maintain germfree mice in colony production long-term had not been demonstrated prior to the studies described herein. Germfree mice have previously been housed in IsoIVC-P for 12 weeks (12) but not incorporating HPV use.

That IsoIVC-P and the QUBE can be used together to support multiple concurrent germfree and gnotobiotic murine cohorts simultaneously in the same room on the same rack without contamination of germfree colonies or complication of germfree or gnotobiotic experimental cohorts had not been demonstrated prior to the studies described herein.

### Combined Use of IsoIVC-P and QUBE

Interiors of all IsoIVC-P and supplies are prepared as above, by autoclave sterilization.

Germfree mice can be received from commercial or core sources in a Taconic shipper, exteriors of which are sterilized by a QUBE HPV loaded cycle, and then unloaded successfully in the QUBE, with mice transferred into sterile IsoIVC-P while retaining germfree status, confirmed by absence of PCR amplicons for bacterial 16srRNA and lack of growth in aerobic, anaerobic, or fungal conditions.

Germfree status of mice in colony production is confirmed by at least weekly documented absence of amplicons using broad nonspecific bacterial primers for 16srRNA and absence of growth in aerobic, anaerobic, and fungal cultures.

Before opening mouse-occupied IsoIVC-P, exteriors of the sealed, mouse-occupied IsoIVC-P and all husbandry and procedural supplies and equipment are first sterilized using a QUBE HPV (QHPV) loaded cycle before husbandry or use.

Only occupied IsoIVC-P that house mice with identical germfree or gnotobiotic consortia are entered together into the QUBE together.

To confirm longitudinally continued germfree and gnotobiotic status, specimens are collected from mice whenever IsoIVC-P are opened inside the aseptic work environment of the QUBE, at least weekly.

We have confirmed QUBE receipt of germfree mice in a Taconic shipper, transferred into sterile IsoIVC-P, while both the QHPV-sterilized Taconic shipper and IsoIVC-P are inside the QUBE aseptic work environment.

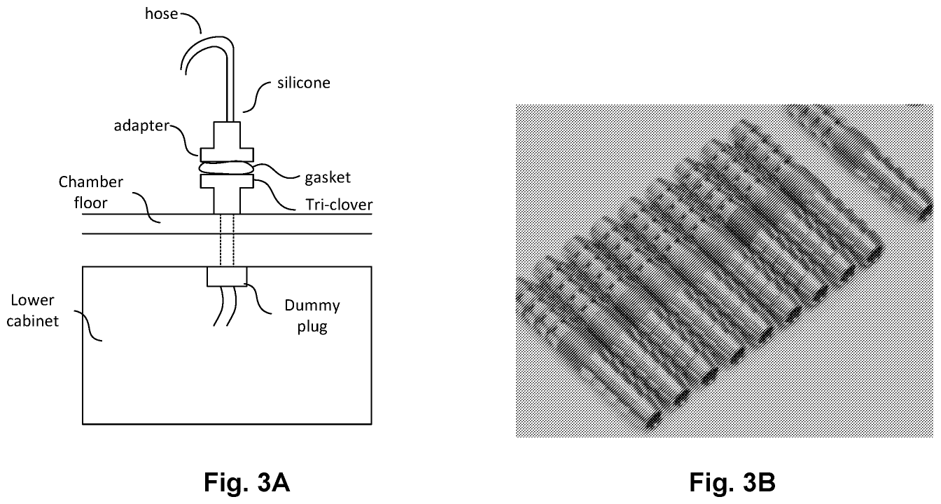

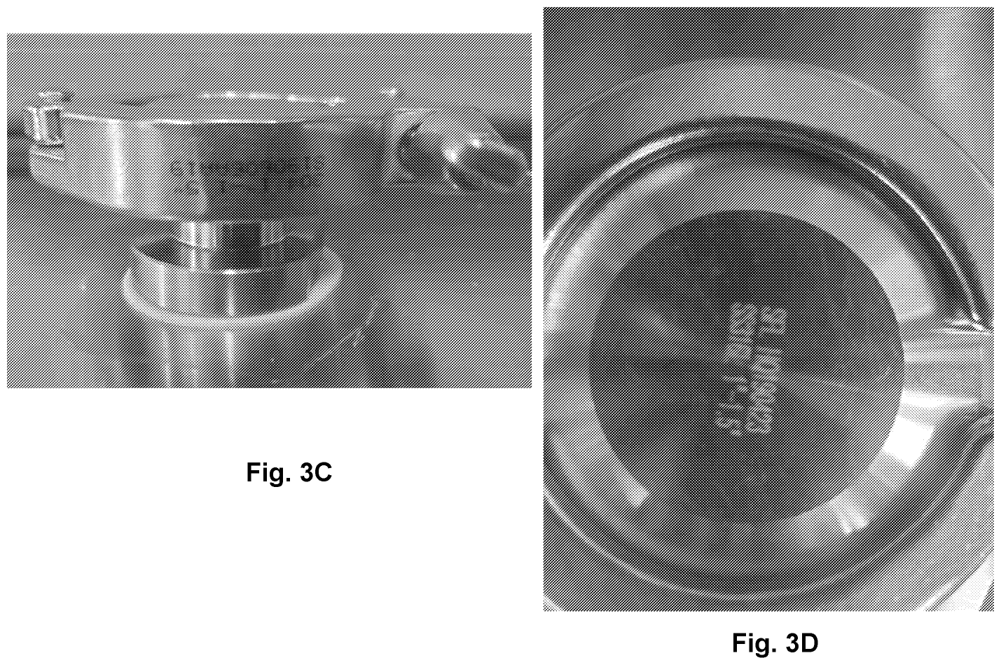

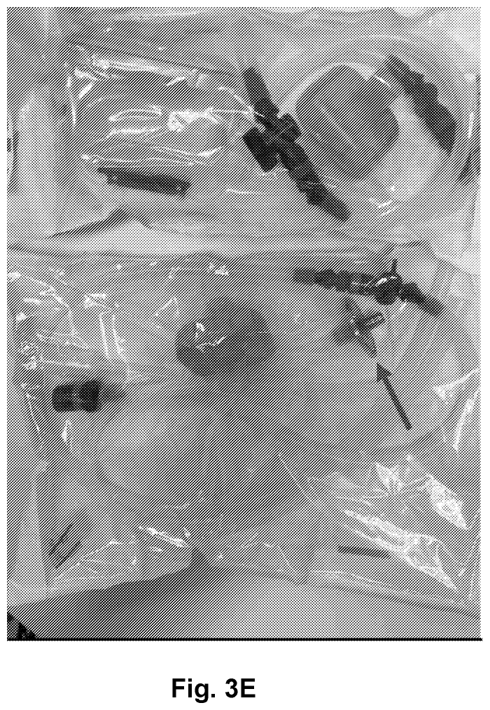

Germfree Isoflurane Anesthesia methods of germfree mice inside the QUBE have also been developed using a tri-clover port adapter and two (2) Pall MGF50 breathing system filters in the anesthesia line, one just prior to the induction box and rodent-specific facemask, and the other just after the vaporizer outside the QUBE. By including the tri-clover port adapter on the isolator and breathing system filters in the anesthesia line, germfree mice can be anesthetized without contamination, allowing for procedures to be performed on the anesthetized germfree or gnotobiotic mice while inside the isolator. FIG. 3A shows that oxygen-bearing isoflurane as an inhalant general anesthetic enters the QUBE via a dummy plug connection that passes through the QUBE chamber floor, through a tri-clover port, through a tri-clover adapter fashioned with a two-sided barbed connector, through the Pall filters, and is delivered to the mouse in either an induction chamber or placed at a rodent-specific anesthetic facemask. FIG. 3B shows the two-sided barbed connector. FIGS. 3C and 3D show the tri-clover port. FIG. 3E shows the tri-clover adapter (arrow), the two Pall filters, the induction box, the facemask, and the connecting tubing.

The Pall MGF50 filter is manufactured to prevent the introduction of microorganisms or airborne contaminates in human clinical medical gas lines (e.g., excluding Human Influenza A, H1N5, HIV, Hepatitis C, Mycobacterium tuberculosis, and pathogenic prion proteins).

Germfree Colony Production Using IsoIVC-P and the QUBE for >8 months, two inbred lines (i.e., C57BL/6, BALB/C) of germfree mice imported from Taconic in a germfree shipper have been maintained in colony production with no 16srRNA amplicons, and no aerobic, anaerobic, or fungal growth, with at least weekly testing.

Use of IsoIVC-P and QUBE together ensure longitudinally that germfree mice remain a clean preclinical platform.

Germfree BALB/c and C57BL/6 murine colonies produced as progeny decedents of the founder germfree mice received from Taconic were documented for >8 months as devoid of microorganisms with no 16srRNA amplicons, and no aerobic, anaerobic, or fungal growth, with at least weekly testing.

Germfree BALB/c and C57BL/6 colonies were also confirmed as germfree using shotgun genomic sequencing as having <1.0% bacterial/microbial reads matching sequences in the Transnetyx One Codex database, with >99% host eukaryotic mouse DNA reads.

Concurrent Germfree & Gnotobiotic Experimental Cohorts on study, housed on the same IsoIVC-P rack as germfree colony mice, were also assessed with 44 BALB/c mice monitored longitudinally, 22 males & 22 females, prior to and after being administered by weekly gavage either sterile saline, or one of two murine fecal slurries, and with each administered HER2-positive murine breast cancer tumor cells by intramammary injection to document whether infrastructure can ensure retention of separate germfree colonies and germfree or gnotobiotic experimental cohorts without contamination or complication of either experimental cohort or germfree colonies housed on the same rack, in the same room, longitudinally. Some experimental cohorts were also administered either or in combination anti-semaphorin4D monoclonal antibodies by intraperitoneal injection, and/or HER2-pulsed dendritic cells by intra-tumoral injection, and all mice were weighed and tumors measured twice weekly. All procedures were performed in the QUBE aseptic work environment.

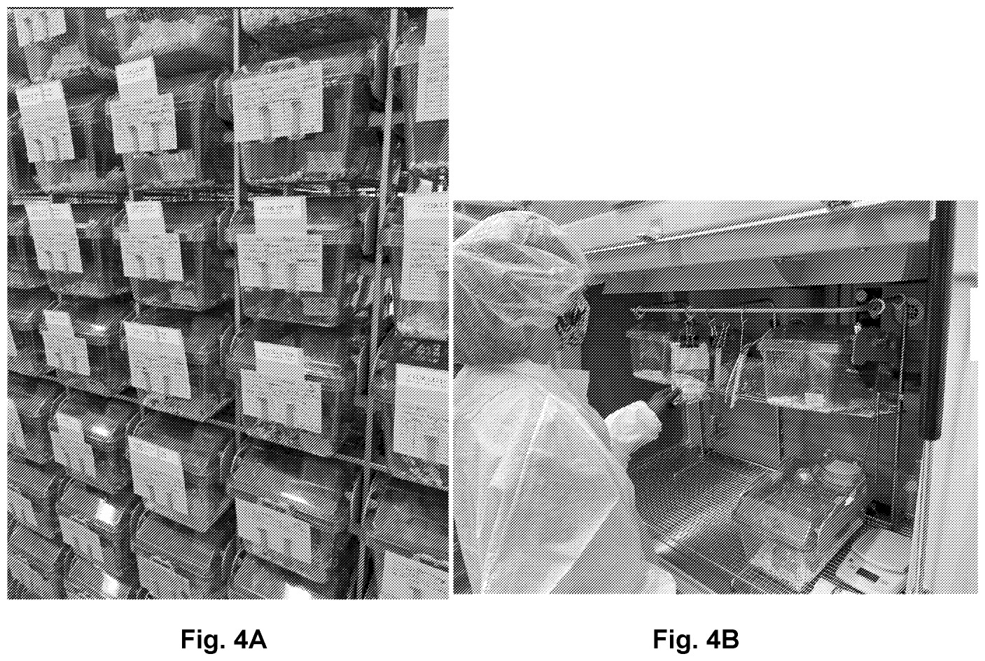

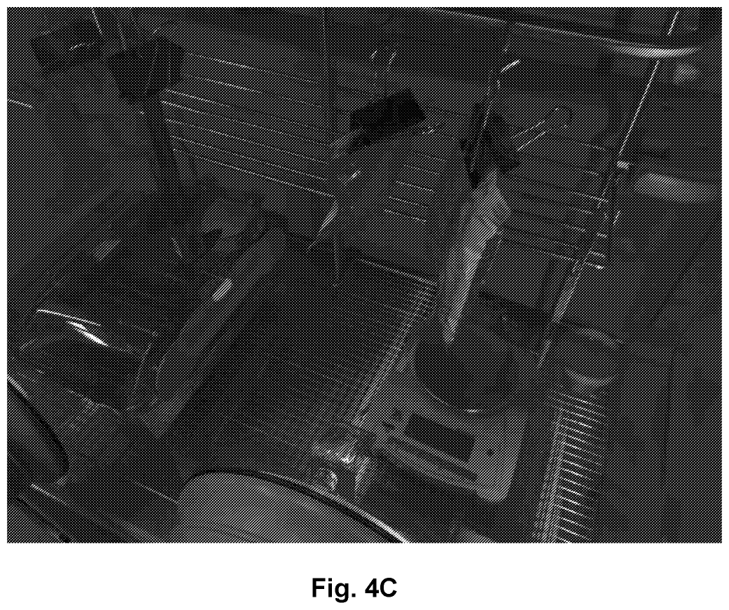

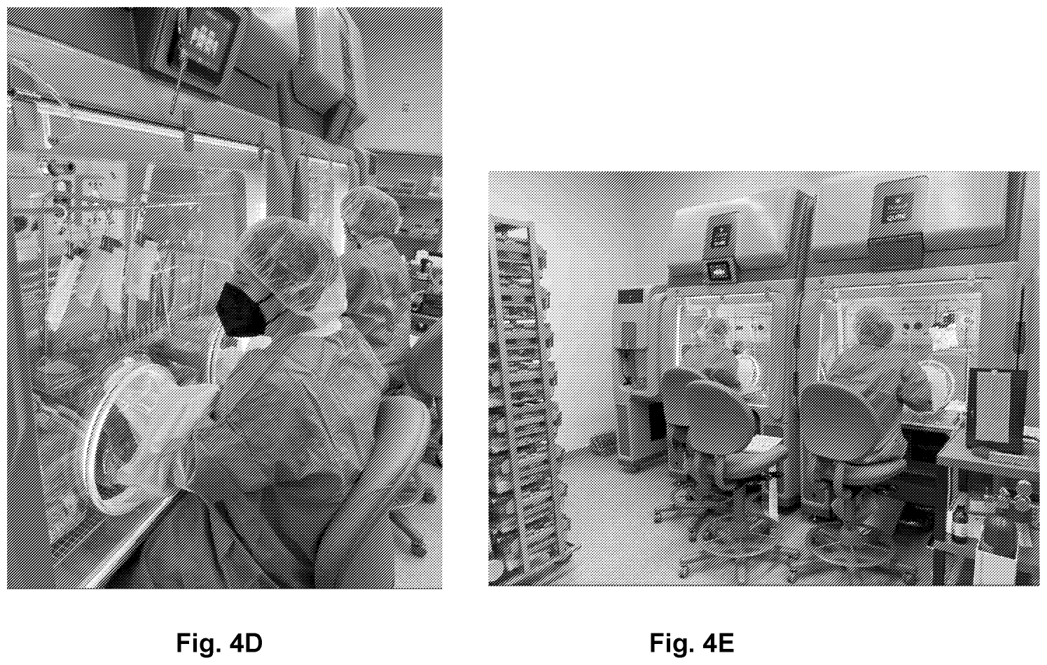

The colony and experimental mice associated with different microbial consortia are housed in separate IsoIVC-P primary enclosures, same rack, same room, all handled in a single QUBE glovebox isolator with integral hydrogen peroxide vaporizer. FIGS. 4A-4E show the concurrent germfree and gnotobiotic systems and methods in use. FIG. 4A shows a rack of IsoIVC-P in use, occupied by germfree colony mice. FIG. 4B shows the process of loading the QUBE with IsoIVC-P and all supplies and instruments needed for mouse use inside the QUBE. FIG. 4C shows supplies and an IsoIVC-P being HPV sterilized during a loaded cycle. FIG. 4D shows the QUBE after a sterilizing loaded cycle during processing mode when IsoIVC-P are opened, and mice are used and handled. FIG. 4E shows both the IsoIVC-P racking system (left) and the QUBE in use (right).

Germfree experimental mice were evaluated using 16srRNA PCR with nonspecific bacterial primers, aerobic, anaerobic, fungal cultures, and using shotgun genomic sequencing, as above.

Mice associated with microflora consortia by weekly gavage of fecal slurry (i.e., gnotobiotic cohorts) were documented using shotgun genomic sequencing as having 25-75% bacterial/microbial reads matching sequences in the Transnetyx One Codex database, with host eukaryotic mouse DNA reads <10%.

To date, only a single IsoIVC-P of intended germfree experimental mice on study administered saline by gavage has been documented with >1.0% bacterial/microbial reads. Paenibacillus macerans, (i.e., bacterium found in plants and soil), and Aneurinibaciluss aneurinilyticus (i.e., bacterium that promotes plant growth) both apparently used in agribusiness for the growth promotion of crops, perhaps making them prevalent in the milled feed or in the cellulose bedding were detected. Insufficient autoclave sterilization of the IsoIVC-P interior and its contents (e.g., feed, bedding) can result in the contamination of germfree mice in any setting. This contamination is the single IsoIVC-P contamination identified in >8 months and was of experimental germfree mice undergoing procedures, so was completed to study endpoint without contamination of other occupied IsoIVC-P, demonstrating the infrastructural ability to confine and strictly limit contamination impact to the affected IsoIVC-P without detriment to the larger inventory.

### Example 1 References

1. G Hecht, C Bar-Nathan, G Milite, I Alon, Y Moshe, L Greenfeld, N Dotsenko, J Suez, M Levy, C A Thaiss, H Dafni, E Elinav, A Harmelin. A simple cage-autonomous method for the maintenance of the barrier status of germ-free mice during experimentation. Lab Anim. October; 48 (4): 292-7 (2014).
2. Jisun Paik, Olesya Pershutkina, Stacey Meeker, Jaehun J Yi, Susan Dowling, Charlie Hsu, Adeline M Hajjar, Lillian Maggio-Price, David A C Beck. Potential for using a hermetically-sealed, positive-pressured isocage system for studies involving germ-free mice outside a flexible-film isolator. Gut Microbes July 4; 6 (4): 255-65 (2015).
3. M Brielmeier, E Mahabir, J R Needham, C Lengger, P Wilhelm, J Schmidt. Microbiological monitoring of laboratory mice and biocontainment in individually ventilated cages: a field study. Lab Anim July; 40 (3): 247-60 (2006).
4. Majbritt Ravn Hufeldt, Dennis S Nielsen, Finn Kvist Vogensen, Tore Midtvedt, Axel Kornerup Hansen. Variation in the gut microbiota of laboratory mice is related to both genetic and environmental factors. Comp Med October; 60 (5): 336-47 (2010).
5. Christa Thoene-Reineke, André Fischer, Christian Friese, Dana Briesemeister, Ulf B Göbel, Thomas Kammertoens, Stefan Bereswill, Markus M Heimesaat. Composition of intestinal microbiota in immune-deficient mice kept in three different housing conditions. PLOS One November 17; 9 (11): e113406 (2014).
6. Randi Lundberg, Martin I Bahl, Tine R Licht, Martin F Toft, Axel K Hansen. Microbiota composition of simultaneously colonized mice housed under either a gnotobiotic isolator or individually ventilated cage regime. Sci Rep February 7; 7:42245 (2017).
7. Schoeb TR, Rahija RJ, Boyd C, Orcutt RP, Eaton KA. Principles of Establishing and Operating a Gnotobiotic Facility. Pg 22-58. In Gnotobiotics, T R Schoeb, K A Eaton, eds, Academic Press, Elsevier, London, UK (2017).
8. Boyd C, Eaton K A, Schoeb T R, Swennes A G, Bickerton H, Parsons B J, Sinclair S, Vowles C. Procedures for Rodents. Pg 66-333. In Gnotobiotics, T R Schoeb, K A Eaton, eds, Academic Press, Elsevier, London, UK (2017).
9. Bier M E. Method of vaporizing multicomponent liquids. U.S. Pat. No. RE.4,642,165 (1987).
10. Grignol G, Eddington D, Karle D, Rickloff J. Chemical and biological aspects of hydrogen peroxide gas, Proceedings of the ISPE Barrier Isolation Technology Conference. Arlington, Virginia, 5-6 Jun. 2000.
11. Hultman C, Hill A, McDonnell G. The physical chemistry of decontamination with gaseous hydrogen peroxide. Pharm Engin 27:22-32 (2007).

### Example 2

### Background

Increasingly, mammals are viewed as comprised of coevolved prokaryotic and eukaryotic parts (1, 2), prokaryotic that is largely bacterial, the oldest living organisms on earth and capable of orchestrating collective behaviors (3), including integrating into and influencing multiple facets of mammalian homeostasis (4-6). Human phenotypes, including why drugs work well for one patient while failing to have an effect on another, are increasingly seen as resulting from complex interactions and combined expressions of multidimensional networks that mutualistically link the host with its microbiota and are affected by diet (4-11). Mice are the model mammal most relied on for preclinical studies with thousands of isogenic lines established that reduce experimental variability and increase the power of studies (7, 8). But genetics is not the only source of variation in these models, and increasingly, preclinical studies rely on an integrated model in which isogenic, germfree mice are recipients of a microbial transplant, either a complete, undefined microbial community (i.e., fecal specimen), or an incomplete, defined microbial consortium, for studies of mechanism and causality in the microbiome. Studies involving microbial transplants to germfree mice have identified a role of the microbiome in a number of human phenotypes including cancer immunotherapeutic responses (12-14), caloric harvest (15), colonization resistance to enteric pathogens (16), neural development (17, 18), and immune modulation (19). Such integrated mouse models require a tractable infrastructure that is accessible, scalable, reliable, and facilitates preclinical research outcomes.

Conventional infrastructure for germfree mice is often a separate core with dedicated staff using flexible-film isolators that enclose static cages, with each isogenic colony housed in redundant isolators, equipment and methods that have remained largely unchanged since a mid-twentieth century effort that determined it is possible to separate the major components of life, microbial from host (20-23). But, isolators enclosing static cages are space inefficient, labor intensive, involve hazardous nebulized liquid sterilants (e.g., peracetic acid), high risk with sterility breaks resulting in loss of the affected isolator's entire murine inventory, necessitating weeks of monitoring a replacement isolator prior to its repopulation with germfree mice, and are problematic from an experimental design and implementation perspective (20-23). Alternative infrastructures have been proposed (24-26), and include a hermetically-sealed, positively pressurized, isolation, individually ventilated cage (IsoIVC-P). The IsoIVC-P is space efficient, with cage level HEPA-filtration, can maintain germfree mice for 12 weeks (27, 28), ensures that defined flora mice remain stable for years (29), prevents microbes in one enclosure from contaminating neighboring enclosures (30), does not increase the variability of complex microbiota (31, 32), but, as in isolators, may contribute to some clustering of the relative abundance of microbes over time (33), and have limitations associated with current methods of IsoIVC-P use involving immersion in a liquid sterilant dunk tank and procedural cabinets with open sashes.

The QUBE is an aseptic workstation, a rigid, glove-box isolator with an integral hydrogen peroxide (H₂O₂) vaporizer, designed for the pharmaceutical and semiconductor industries, and used for the hydrogen peroxide vapor (HPV) decontamination of a load, after which a unidirectional HEPA-filtered airflow working zone is maintained during “processing mode” that meets the cleanroom classification requirements of ISO 14644-1 Class 5 (<3,520 airborne particles ≥0.5 μm). Using room-based H₂O₂ generators, HPV is also used to decontaminate vivarium settings to mitigate opportunistic agents that invalidate studies involving immunodeficient mice (34-38), and clinical settings to mitigate nosocomial agents (39-42). HPV involves the conversion of liquid concentrate 35% H₂O₂ to a gaseous phase by flash vaporization (43-45). During sterilization, HPV is dispersed in a dry air stream onto all exposed surfaces sealed within an airspace. In its gaseous phase, even low HPV concentrations of 0.1 mg/L (10 ppm) are rapidly, broadly antimicrobial and sporicidal through oxidation, with a 6-log reduction of microbes occurring in minutes at 150 to 400 ppm, after which HPV degrades to water and oxygen, with negligible contribution to atmospheric oxygen and no condensation (43-45). Due to the dry nature of the HPV decontaminating cycle and high material compatibility, HPV is safe on many surfaces, including electronics, metals, plastics, and elastomers, resulting in no substantive change in physical or chemical properties (45). After HPV surface sterilization, during the aeration phase, HPV is degraded to below the long-term exposure limit (LTEL) 8-hour time weighted average (TWA) of <1 ppm, a process accelerated by an aeration catalytic accelerator.

Here, we combined technologies previously unaware of one another, the IsoIVC-P and QUBE systems, and developed methods that are highly tractable and scalable, capable of long-term germfree murine colony maintenance, and concurrent germfree and gnotobiotic mouse model throughput, in the same vivarium room, a highly space-efficient practice heretofore not attempted. Rather than enclosing each germfree isogenic colony in static cages in redundant isolators in separate rooms, and bringing cylinders of sterile supplies to each isolator, as is traditionally done, we used the IsoIVC-P for all germfree and gnotobiotic murine housing, and took each microbially-similar cage to the QUBE for HPV exterior surface sterilization prior to opening it and mouse use. Rather than segregating germfree and gnotobiotic murine inventories into separate rooms, as is traditionally done, we tested whether the IsoIVC-P and QUBE systems were robust enough to support concurrent housing and use of germfree and gnotobiotic mice on the same rack in the same room. Herein we demonstrate that this heretofore undisclosed combination of preclinical infrastructure has sufficient integrity, reproducibility, and throughput to maintain multiple germfree isogenic colonies for >74 weeks with a performance metric equivalent to the isolator “gold standard”, while concurrently supporting multiple studies involving germfree and gnotobiotic experimental cohorts each associated with unique microbial consortia, on the same rack in the same room, to study end points without alteration, thereby improving microbiome research accessibility, space efficiencies, eliminating hazards, and reducing risks posed by traditional infrastructure.

### Results

Safety & Efficacy: We tested how long the hermetically-sealed IsoIVC-P with an interior volume of 6,750 cm3 providing 1,407 ml of oxygen can be left undocked and unventilated and be supportive of murine life. We assumed 5 adult mice would consume 12 ml of oxygen per minute, calculated that approximately 1 hour and 54 minutes of oxygen is available to 5 adult mice in the unventilated IsoIVC-P, and confirmed by direct observation that five 25-30-gram C57BL/6 male mice were very active and unaffected while sealed in an undocked IsoIVC-P for >90 minutes. Hence, we adopted 90 minutes as our safety threshold for how long a murine-occupied IsoIVC-P can be left undocked and unventilated.

To test without animals the safety of the IsoIVC-P while being HPV decontaminated in the QUBE, and the efficacy of the “QHPV loaded cycle” to decontaminate IsoIVC-P exterior surfaces, we used a QUBE model M35, comprised of four chambers, including two large glove-box chambers which serve as the work environment for one or two personnel during “processing mode” when contents are used, after having been HPV sterilized. The left glove-box chamber is referred to as the “QHPV” module and is equipped with the HPV generator and catalytic accelerator, into which contents are loaded via its front top-hinged sash window and HPV sterilized. The right glove-box chamber is referred to as the “QEXT” or extension module, into which HPV sterilized contents can be shared during processing mode via an interior door connecting the QHPV and QEXT. Two decontamination cycles are possible with the M35, either of the entire system empty with its three interior doors open interconnecting all four chambers, known as a “system empty cycle”, or of a load placed within the QHPV, known as a “QHPV loaded cycle”. A QHPV loaded cycle lasts 40-45 minutes, half the time an occupied IsoIVC-P can be safely left undocked and unventilated with mice.

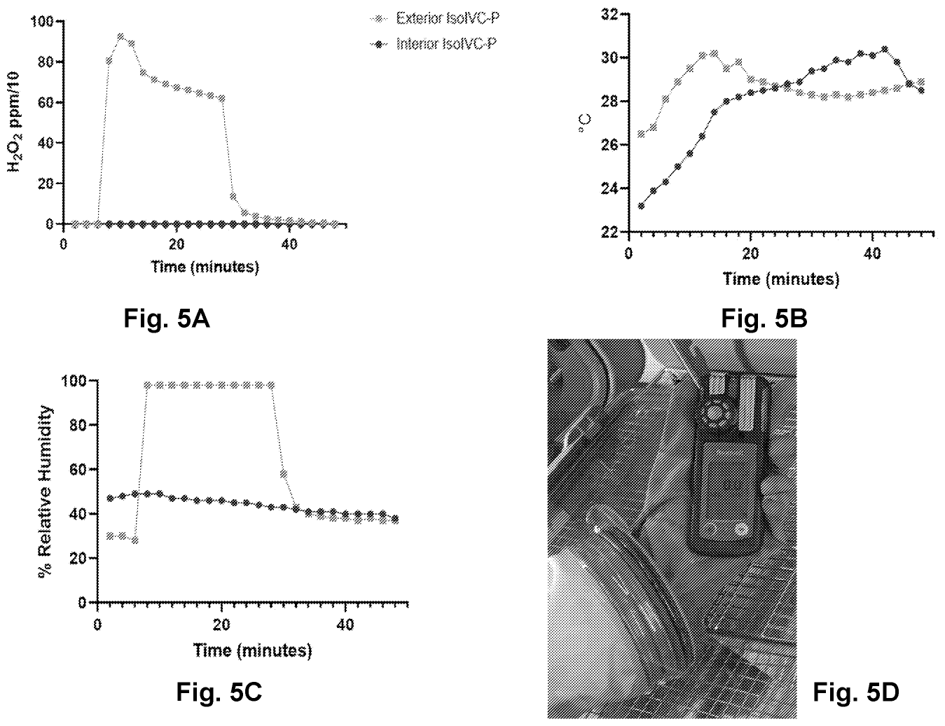

To test without animals whether the QHPV loaded cycle is safe and effective for preclinical applications, providing sterilization of IsoIVC-P exterior surfaces while unaffecting IsoIVC-P interiors wherein mice would later reside, fifty-six (56) biological and chemical indicators of HPV were placed inside and outside of an unoccupied IsoIVC-P and germfree shipper and exposed to a QHPV loaded cycle in the M35. Each of the two vents of the germfree shipper were covered with a flexible port cap. All biological and chemical indicators of HPV placed on exterior surfaces of the IsoIVC-P and germfree shipper showed a >6 log microbial deactivating level of HPV, while all indicators placed in the interiors of the IsoIVC-P and germfree shipper did not detect any HPV. Conditions inside and outside of the IsoIVC-P during a QHPV loaded cycle indicated an IsoIVC-P exterior sterilizing effect by HPV, while the IsoIVC-P interior experienced only a modest increase in ambient temperature (FIGS. 5A-5C).

Ambient conditions during the HPV loaded cycle were documented inside the sealed, unoccupied IsoIVC-P wherein mice would later reside, and inside the QHPV module but outside of the IsoIVC-P. During the HPV loaded cycle, inside the IsoIVC-P, no HPV was detected, relative humidity was negligibly affected, and temperature rose ˜7° C. After the QHPV loaded cycle, that HPV had degraded to an undetectable <1 ppm level inside the QHPV module was confirmed using a handheld low-level hydrogen peroxide monitor placed earlier inside an IsoIVC-P (FIG. 5D).

After completing a QHPV loaded cycle, sterile swabs were used to collect specimens from the exterior surfaces of the unoccupied IsoIVC-P and other surfaces inside the QHPV and these specimens subjected to polymerase chain reaction (PCR) for bacterial 16s rRNA and to microbiological cultures. After an QHPV loaded cycle, none of the 18 specimens collected from IsoIVC-P exterior surfaces were positive by PCR bacterial 16s rRNA or culture for microbial growth.

Germfree & Defined Flora Importation: Germfree mice were imported, BALB/c mice on Aug. 10, 2021, C57BL/6 mice on Oct. 6, 2021, and Swiss Webster mice on Apr. 27, 2022, and each shipment arrived in a germfree shipper in 3 static cages. A system empty cycle was completed to prepare the QUBE system for unpacking the germfree shipper and for housing the germfree mice in the IsoIVC-P. After the system empty cycle, autoclave sterilized IsoIVC-P with feed, bedding, and enrichments were subjected to a QHPV loaded cycle, then passed into the QEXT for mouse receipt. Then the germfree shipper was subjected to a QHPV loaded cycle. It was assumed that the overlapping plastic flaps of the port caps used to seal the vents of the germfree shipper would likely hide microbes from HPV sterilization, so only the smooth plastic underbelly of the shipper was handled, opened using a sterile scalpel blade, and the 3 static cages holding mice removed from the shipper and passed into the QEXT for mouse transfer to IsoIVC-P for housing. Imported germfree mice were mated in IsoIVC-P for colony production, confirmed to be absent of immigrant microbes by PCR for bacterial 16s rRNA and microbiological cultures, and similarly monitored at least every third week when cages were changed.

Altered Schaedler Flora (ASF) mice were also similarly imported on Jun. 29, 2022, confirmed by shotgun sequencing to be colonized with ASF356 Clostridium sp., ASF360 Lactobacillus intestinalis, ASF361 Lactobacillus murinus, ASF457 Mucispirillum schaedleri, ASF492 Eubacterium plexicaudatum, ASF500 Pseudofalvonifractor sp., ASF502 Schaedlerella arabinosiphila, and ASF519 Parabacteroides goldsteinii, similarly unpacked, mated, and housed for defined flora colony production on the same rack.

QHPV Processing Mode Environmental Monitoring: Since every IsoIVC-P housing either germfree or gnotobiotic mice would first be decontaminated in and then opened for mouse use in the same QHPV, we were interested in documenting the air particle load cleanup of the QHPV module during animal handling, FMT gavage administration to mice, and the transfer of mice from soiled to clean IsoIVC-P during processing mode. Sixteen (16) germfree female 8-10-week-old C57BL/6 mice were assigned to one of four cohorts and every other week were gavage-administered 100 μl of either sterile PBS, ASF FMT, or one of two unique murine specific pathogen-free (SPF) FMTs for 6 weeks, and subjected to microbiota shotgun sequencing of fecal pellets every 2 weeks. Each cohort was exposed to a QHPV loaded cycle and handled separately in the QHPV module, but the sequence of cohort use was altered each week so that the germfree mice receiving PBS by gavage were handled both as the first cohort and the last cohort in the QHPV module during the 6-week study.

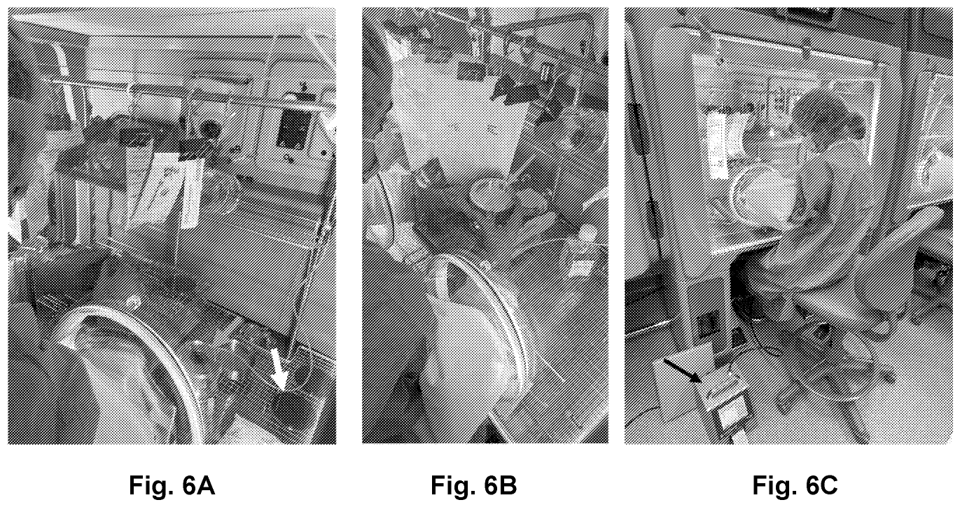

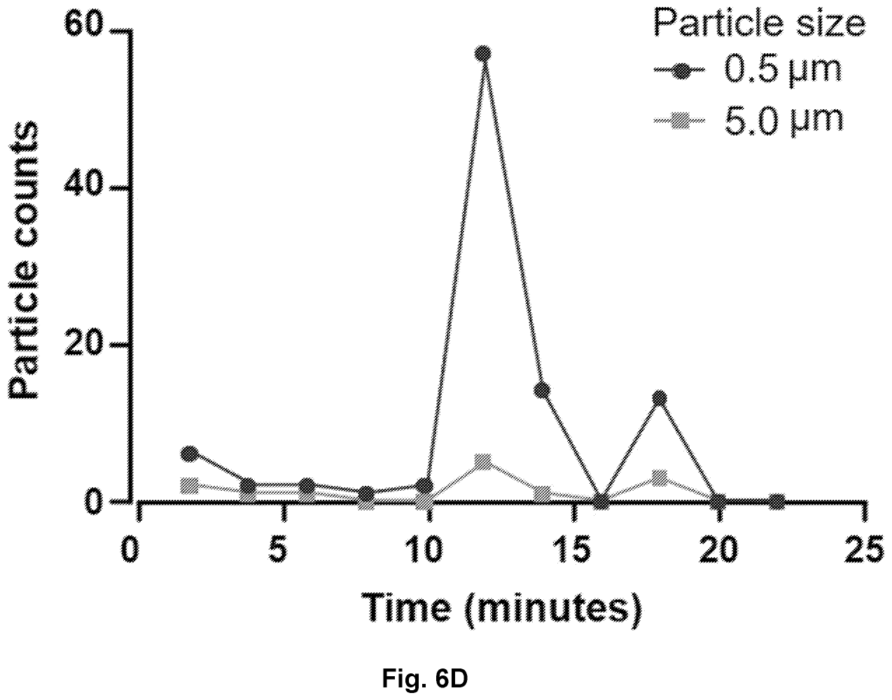

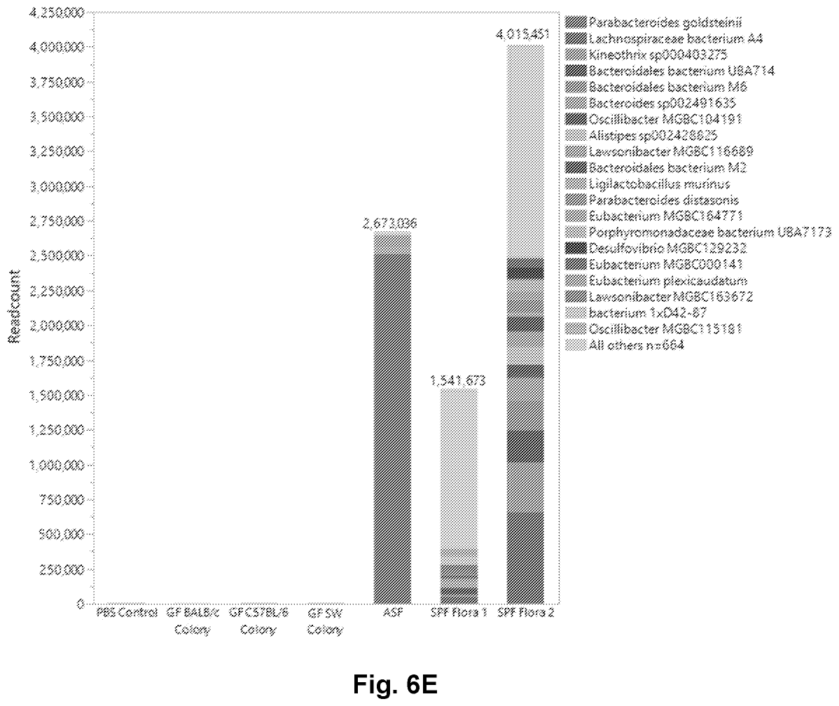

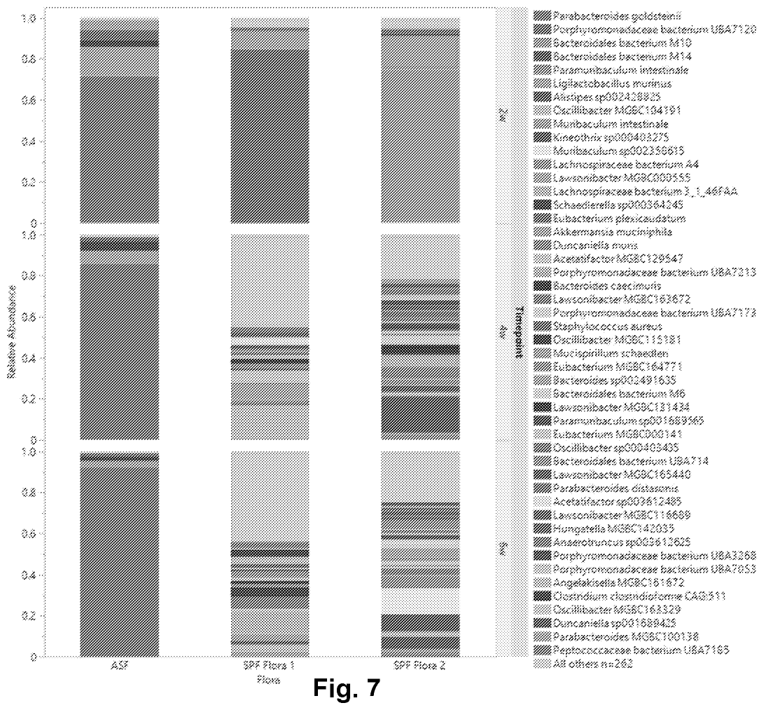

Live airborne microbes were recovered in room air with the QHPV front sash open and in the HPV sterilized QHPV module during processing mode using an environmental culture plate set in an active air sampler (FIG. 6A-B), while total airborne particles were counted using a light-scatter airborne particle counter (FIG. 6C). Microbes recovered in room air included Microbacterium paraoxydans, Micrococcus lutues, Micrococcus sp., Staphylococcus epidermis, Streptococcus parasanguinis, Corynebacterium amycolatum, Staphylococcus warneri, Bacillus subtilis, and Staphylococcus sp., and an average 6,718 particles ≥0.5 μm were present. In contrast, during QHPV processing mode, despite the gavage material administered, the M35 unidirectional airflow of 573 m3/h through HEPA filters maintained requirements of ISO 14644-1 Class 5 with low airborne particle counts detected in all twelve QHPV processing modes (FIG. 6D). No live microbes were recovered when handling germfree mice and administering sterile PBS by gavage. Ligilactobacillus murinus was the sole agent recovered during processing mode of ASF FMT administered mice. Staphylococcus xylosus, Enterococcus faecalis, Enteroccus gallinarum were recovered during processing mode of SPF FMT administered mice. Shotgun sequencing of fecal pellets after 6 weeks, indicated no immigrant microbes of GF colony mice or PBS controls with total readcounts of <10,000, compared to >1.5 million for each of the FMT colonized cohorts housed on the same rack (FIG. 6E). In addition to confirmed maintenance of germfree status, we were able to show that colonization with distinct, discernable flora remained distinct for the duration of the 6-week study (FIG. 7). The richness, evenness, and diversity of the ASF-populated mice remained low, while each increased from week 1 to 4 then stabilized in the two SPF-populated mice.

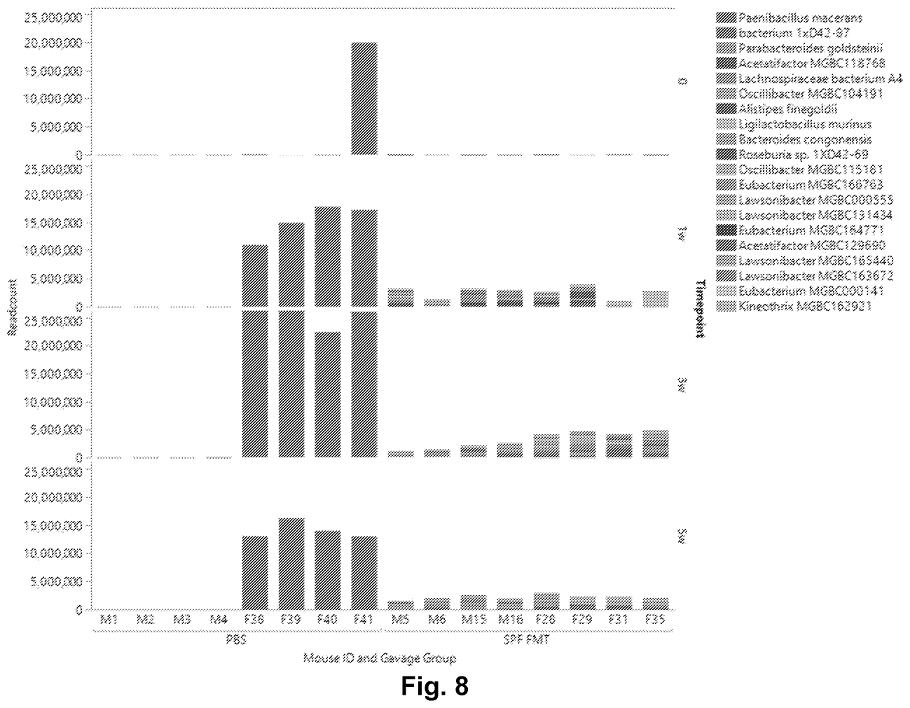

Microbial Colonization: Since the infrastructure would be expected to support large experimental cohorts segregated into multiple IsoIVC-P, we were interested in determining the fecal microbiota in SPF FMT gavage-administered mice for consistency of colonization. Forty-four, 22 male and 22 female, germfree 8-10-week-old BALB/c mice were assigned to one of two cohorts, with 12 mice, 6 male and 6 female mice, administered sterile PBS by gavage, and 32 mice administered SPF FMT by gavage, each weekly. PCR for bacterial 16s rRNA and microbial cultures and shotgun sequencing of fecal pellets of each mouse was performed prior to study, and fecal microbiota shotgun sequencing of each mouse performed at weeks 1, 3, and 5. Prior to study, a mono-contaminant of Paenibacillus macerans, apparently of feed origin, was identified and cultured from a single female mouse in the PBS-administered cohort (FIG. 4, top). This mono-contaminant spread only to other germfree female mice in the same cohort handled during the same processing mode. In contrast, germfree male PBS gavage administered mice and germfree colony mice housed on the same rack nearby, and processed separately in the QUBE, remained germfree (FIG. 8). Further, no microbes from the SPF FMT mice contaminated germfree mice of the PBS administered cohorts, or germfree colony mice housed on the same rack. Comparisons of colonization showed that the SPF FMT colonized each individual mouse of the FMT administered cohort similarly (i.e., no individual variability), whether mice were housed in the same or in a different IsoIVC-P (i.e., no cage effect).

Among SPF FMT gavage administered mice, the greatest number of OTUs that were different both within and between groups was from weeks 1 and 3 and the fewest between weeks 3 and 5, suggesting stabilization of the microbiota transfer over time. Overall, female (n=44 samples) and male (n=40 samples) mice administered SPF FMT had a differing relative abundance of three OTUs: Kineothrix sp000403275 (FDR p-value=0.040; 0-22.8, 0.9 and 0-16.2, 2.7 [min-max, median of female and male mice, respectively]), Bacteroides uniformis (FDR p-value=0.042; 0-37.5, 0 and 0-0, 0), and Roseburia sp. 1XD42-69 (FDR p-value=0.042; 0-3.1, 0.7 and 0.1-4.4, 1.0). Evenness and diversity did not differ between weeks 1 and 3 (p=0.064 and 0.313, respectively), but did differ between weeks 1 and 5 (p=0.002 and 0.006) and weeks 3 and 5 (p=0.005 and 0.016). Richness differed between each timepoint (p<0.05).

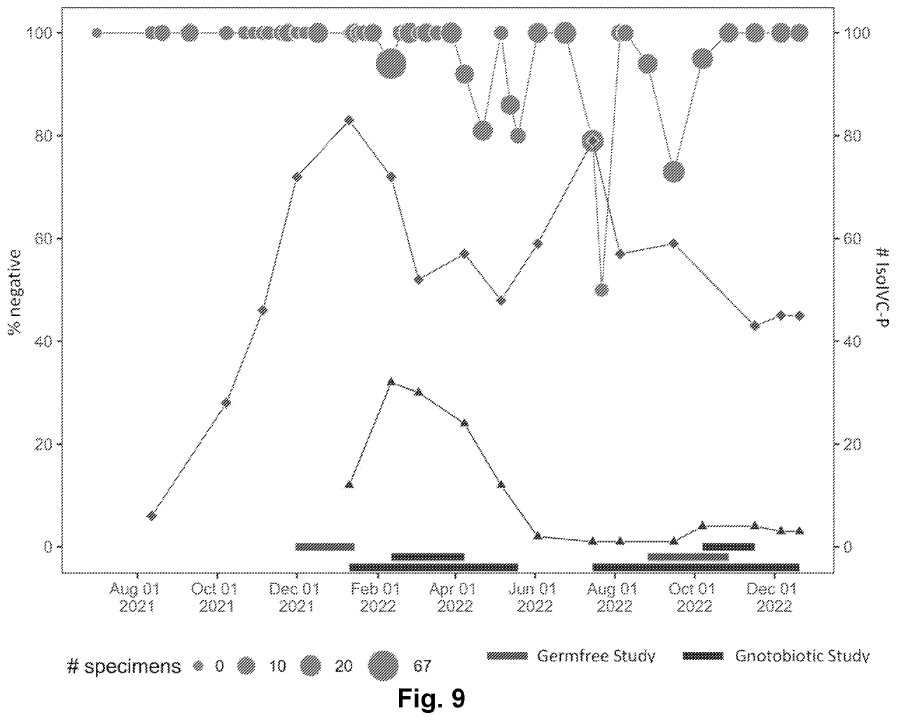

Germfree Colony Maintenance: Germfree colony mice were maintained in IsoIVC-P for >74 weeks. Fecal pellets were collected and pooled from each IsoIVC-P after each QHPV loaded cycle and confirmed to be absent of immigrant microbes by PCR for bacterial 16s rRNA and microbiological cultures at least every third week when cages were changed. Of the 468 specimens tested, 443 or 95% were PCR and culture negative for immigrant microbes. Of the 1, 120 occupied IsoIVC-P tested, 1,070 or 96% were PCR and culture negative for immigrant microbes. This while 126 IsoIVC-P were being used to house FMT experimental mice or ASF defined flora colony mice in the same room, processed through the same QUBE. Five breaks in sterility were experienced in the germfree colony, causing 50 IsoIVC-P of germfree colony mice to be reassigned to a SPF murine housing room and used for technical training purposes, with Enterococcus gallinarum, apparently of murine fecal origin, recovered on culture from four of the sterility breaks, and Paenibacillus macerans recovered from one sterility break, as described above. Overall, during the >74 weeks of evaluation, a performance metric of 0.0001 sterility breaks/IsoIVC-P/week was experienced, while the IsoIVC-P and QUBE systems were used exclusively for >1,379,693 germfree mouse-days, and for >394, 198 germfree cage-days (FIG. 9).

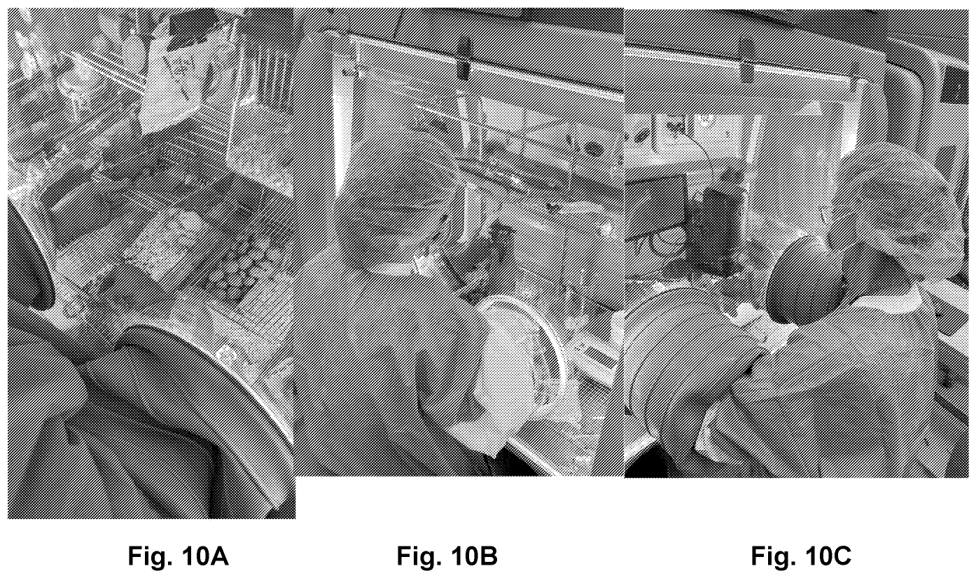

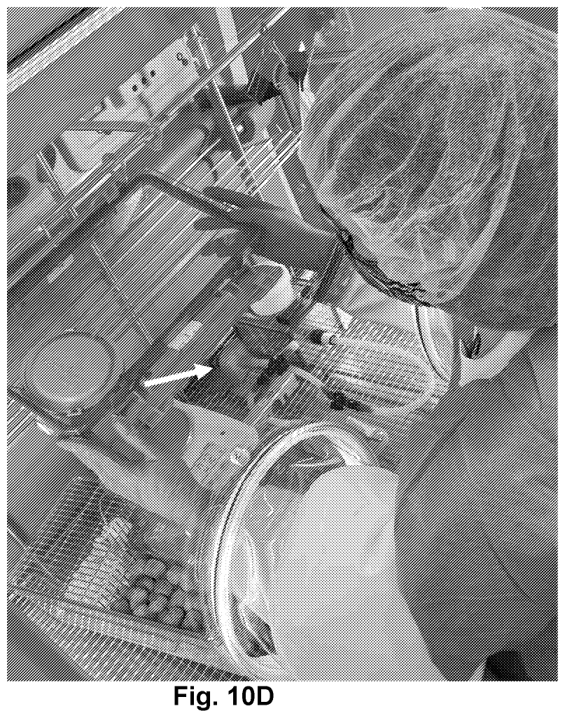

Germfree & Gnotobiotic Studies Supported: During the >74-week study, while conducting the assessments described above, three additional studies were supported by the infrastructure described here. Two studies were completed in germfree mice to establish tumor growth curves for three cancer cell lines of interest (i.e., mEER and MTE squamous carcinoma cells grown heterotopically in syngeneic, subcutaneous flank models, and TUBO mammary carcinoma cells grown in a syngeneic, orthotopic model). A third study was completed using the syngeneic, orthotopic HER2+TUBO breast cancer mouse model involving 8 cohorts of 10 BALB/c mice, variously administered treatments of weekly SPF FMT, weekly intratumor HER2-activated dendritic cells, and weekly intraperitoneal anti-semaphorin-4D monoclonal antibody, while monitoring intramammary tumor growth twice weekly (FIG. 10). Each of these three additional studies was completed without alteration in the same room using the same infrastructure while it was used for germfree colony production and the assessments described above.

### Discussion

The disclosed infrastructure introduces HPV sterilization to the germfree setting, traditionally reliant on liquid sterilants (e.g., peracetic acid) and autoclave sterilization. Indeed, of the five sterility breaks experienced, four were attributed to inadequate autoclave sterilization, either of feed, resulting in a Paenibacillus macerans mono-contaminant of a germfree experimental cohort once, or of the change-out IsoIVC-P, resulting in an Enterococcus gallinarum mono-contaminant of germfree colony mice in three cases. That these contaminants spread only to mice in the same QHPV loaded cycle, but not to other germfree cohorts or germfree colony mice on the same rack is a testament to the integrity of the infrastructure developed. After a QHPV loaded cycle, IsoIVC-P exterior surfaces lack evidence of PCR bacterial 16s rRNA or microbial growth, and during processing mode, the QUBE provides a unidirectional HEPA-filtered air particle cleanup, so that even during FMT gavage, low airborne particle counts are maintained. Still, live microbes can be introduced into the QEXT air space during processing mode (e.g., during FMT gavage), and since workflow in the model M35 can be from left to right with the QEXT kept in “aseptic hold” and the QHPV used to decontaminate multiple, successive loads that are then passed into the QEXT for processing, one of the five sterility breaks experienced was attributed to inappropriate sequence processing through the QEXT. As a result, we learned that prior to QEXT use, a “system empty cycle” must first be run sterilizing the QEXT between each successive microbially unique cohort. The infrastructure supported germfree colony mice for >74 weeks, with a performance metric of 0.0001 sterility breaks/IsoIVC-P/week experienced, with 96% of germfree colony IsoIVC-P negative by PCR and culture for immigrant microbes, while 126 neighboring IsoIVC-P were used to house gnotobiotic mice. Conventional infrastructure for only germfree colony mice in redundant isolators in separate rooms experiences a performance metric of 0.0052 sterility breaks/isolator/week (48), and when a break in sterility is experienced, requires 3-6 weeks of monitoring a new isolator prior to its reuse (49). In contrast, the infrastructure here provides greater security with multiple redundancies of each germfree isogenic line in each IsoIVC-P. When a break in sterility is experienced, mice in the affected IsoIVC-P are reassigned to training purposes and relocated to a neighboring SPF murine housing room, with little threat of loss to the germfree murine line. If a traditional colony isolator is assumed to enclose 50 static cages of germfree mice, then the “gold standard” isolator performance metric would be 0.0001 sterility breaks/husbandry unit/week, identical to the performance metric of the infrastructure described here.

Conventional infrastructure is inefficient and inaccessible, often a separate core with dedicated staff, with redundant germfree colony isolators in separate rooms, germfree experimental cohorts in yet another isolator, and gnotobiotic mice housed and used in a separate, third room. In contrast, the infrastructure described here was established in a single 316 net square feet (nsf) vivarium room and supported by staff also responsible for the care of other species. If the murine inventories and experiments described here were to be implemented utilizing traditional infrastructure, substantially more, approximately 3-times as much, space and equipment would be required, including three vivarium rooms, eight husbandry isolators (i.e., four husbandry isolators in each redundant germfree colony room), multiple double-tiered germfree experimental isolators, at least one surgical/procedural isolator, and gnotobiotic housing and use in a separate, third room utilizing IsoIVC-P and an IBS-Isocage biosafety station.

In addition to limitations in space, staffing, and expertise that institutions face when establishing a germfree and gnotobiotic core, it is often difficult to reliably support multiple studies utilizing traditional gnotobiotic housing methods. Researchers are often hesitant to house animals of differing microbial status in the same room and establishing an appropriate handling order and staff schedule can be complicated. A single cross-contamination event can lead to loss of valuable data and necessitate depopulation of the affected colonies. Here, we have shown that multiple gnotobiotic flora can be maintained on the same IsoIVC-P rack, without comingling of microbes. Following FMT, the richness of the fecal microbiota of ASF mice remained low and unchanged, while the evenness and diversity remained low. This, despite the concurrently housed SPF-gavaged mice showing a typical pattern of significant increases to much higher levels of evenness and diversity as the normal, robust microbiota established and stabilized, further highlighting that there were no crossover events with either of the SPF floras.

Here, we demonstrate that the methods provided herein and combined infrastructure involving the IsoIVC-P and QUBE systems can maintain germfree colony mice long-term, and ensures that ASF defined flora mice and SPF FMT administered experimental mice do not contaminate germfree mice in neighboring IsoIVC-P. Even following a contamination event, our combination of technologies allowed research to continue in the same space, with no downtime due to decontamination and follow-up testing of equipment and did not necessitate depopulation of valuable research animals. The infrastructure does not increase the variability of FMT complex microbiota, has lower risk and fewer hazards than traditional infrastructure, with no liquid sterilant required. It is space efficient and versatile, permitting germfree husbandry, inhalational anesthesia, and microsurgical procedures all in the same footprint. It is highly reliable and more accessible, increasing the potential for new discoveries of the microbiome.

### Methods

Setting: Evaluations were conducted in a 316 nsf germfree and gnotobiotic mouse housing room of a 18,695 nsf vivarium of the accredited (AAALAC #000434) and assured (PHS/NIH/OLAW #D16-00589) animal care and use program of the University of South Florida, Tampa, Florida, which excludes a broad spectrum of murine pathogens, including murine norovirus, Helicobacter spp., Syphacia spp., Aspiculuris tetraptera, parainfluenza virus type 1 (Sendai virus), mouse coronavirus (mouse hepatitis virus), Mycoplasma pulmonis, paramyxovirus (pneumonia virus of mice), parvoviruses (minute virus of mice and mouse parvovirus type 1), poliovirus (Theiler murine encephalomyelitis virus), reovirus type 3, Lymphocytic choriomeningitis virus, mouse adenovirus type 1 and 2, poxvirus (Ectromelia), rotavirus (epizootic diarrhea of infant mice virus), papovavirus (K virus), Hantaan virus, cilia-associated respiratory bacillus, Clostridium piliforme (Tyzzer disease), Encephalitozoon cuniculi, and Corynebacterium bovis. In other rooms, the vivarium houses and uses SPF mice and rats, and has an automated zebrafish aquatics system.

IsoIVC-P: IsoIVC-P mouse housing racks (Tecniplast, Buguggiate, Italy) were used in a double-sided rack configuration, with each rack holding 95 IsoIVC-P, and each IsoIVC-P with a cage-level HEPA filter. Each double-sided rack was ventilated and exhausted by an electronic air handling unit (AHU). IsoIVC-P cages and bottles, racks and plenums were washed in a mechanized washer (Atlantis or Arcadia models, Tecniplast) ensuring 180° F. final rinse temperature. Exterior and accessible interior surfaces, prefilter chambers, and connecting ports of the AHU were hand-sanitized with Oxivir Tb (Diversey, Sturtevant, WI) and then connected to its IsoIVC-P rack and HPV-exposed as previously described (34), in an otherwise empty, sealed, vivarium housing room using a Proteq model H₂O₂ vaporizer and aeration catalytic accelerator (Bioquell, Ecolab, Horsham, PA). After HPV exposure, two IsoIVC-P racks, each with its AHU, was positioned in the 316 nsf germfree and gnotobiotic murine housing room, providing 190 autoclave-sterilized IsoIVC-P, prepared as described below. IsoIVC-P racks were changed every 6 months. IsoIVC-P were changed every 3 weeks. Cage level HEPA filters were changed every 5 autoclave sterilization cycles.

QUBE: The QUBE (Bioquell) is an aseptic workstation with an integral hydrogen peroxide vaporizer, a rigid, glove-box isolator which is loaded via its front top-hinged sash window. The QUBE system is offered in multiple configurations. Herein, we used a QUBE model M1 comprised of a single glove-box chamber, and a QUBE model M35 comprised of four chambers, including two large glove-box chambers which serve as the work environment during “processing mode” (i.e., after HPV sterilization, when contents are being manipulated and used). The large chamber on the left of the M35 is equipped with the HPV generator and catalytic accelerator and is referred to as the “QHPV” module, while the large chamber on the right of the M35 is referred to as the “QEXT” or extension module. The model M1 consists of only a QHPV. Each large chamber of the model M35, both the QHPV and QEXT, are flanked by a smaller chamber, referred to as a material transfer device (QMTD), which is used for exiting processed material via its interlocked doors. The QUBE system is controlled via a control panel on the QHPV. Two decontamination cycles are possible with the model M35. Decontamination can be of the empty system with all its interior doors open interconnecting all four chambers, known as a “system empty cycle”. Alternatively, after a load is placed inside the QHPV via its front sash window, exterior surfaces of the load are decontaminated by a “QHPV loaded cycle”. After either decontamination cycle, the QHPV and QEXT are each considered in “aseptic hold” until its seal is disrupted (e.g., the front sash is opened).

During “processing mode”, after QHPV decontamination, the QUBE system provides a unidirectional airflow working zone that meets the classification requirements of ISO 14644-1 Class 5 (equivalent to EU cGMP Grade A and US FED STD 209E Class 100), an airflow rate of 573 m3/h, and M35 chamber pressure set points of 75 Pa for the QHPV, 50 Pa for the QEXT, and 36 Pa for each of the QMTDs (i.e., a positive pressure gradient from the QHPV to the room). The QHPV, QEXT and QMTD interior doors are fitted with pneumatic inflatable seals, which automatically inflate when the door is manually closed. All model M35 interior doors are interlocked, and each has an indicator light which is either lit continuous green when the door is enabled and can be opened, not illuminated when it is disabled and cannot be opened, flashing red when there is an alarm involving the door seal, or flashing green when an action is required of the door (e.g., “open the door” prior to the start of a system empty cycle, or “close the door” at the end of a system empty cycle). During processing mode, when exiting materials from either the QHPV or QEXT via the adjoining QMTD, only one door of the QMTD can be opened at a time. After opening the QMTD external door to the room, there is a 2-minute clean-up time before the internal door of the same QMTD can be opened again.

Each HPV loaded cycle of the QHPV, either the M1 or M35 models, consists of 5 phases: conditioning, gassing G1, gassing G2, dwell, and aeration. During the initial conditioning phase, the internal temperature of the HPV generator increases so that the 35% concentrate liquid H₂O₂ is flash-vaporized into gaseous HPV. Sensors within the HPV generator stabilize to ambient temperature and humidity, and pumps prime. Once an acceptable internal generator temperature threshold for H₂O₂ flash vaporization has been achieved, the gassing G1 phase begins with a rapid increase in HPV concentration inside the QUBE modules, followed by the gassing G2 phase and a further increase in HPV concentration, which results in rapid, broad antimicrobial and sporicidal activity through oxidation. After the G1 and G2 gassing phases, the dwell phase ensures that peak HPV decontaminating concentrations are maintained for several minutes inside the QUBE modules. During aeration, HPV is converted to water and oxygen by an aeration catalytic accelerator.

During early testing before animal use, chemical and biological indicators of HPV were placed inside and outside of a sealed Iso-IVC-P and sealed germfree shipper (Class Biologically Clean, Madison WI). Each of the two vents on the germfree shipper were covered with a flexible port cap (Class Biologically Clean). Biological indicators (Bioquell) were used to detect HPV levels capable of a 6-log reduction in microorganismal growth from Geobacillus stearothermophilus spores sealed inside pouches that were afterwards incubated in trypticase soy broth at 60° C. for 7 days. Chemical indicators (Bioquell) provided a semiquantitative visual indication of 2-log, 4-log, 6-log, or greater than 6-log microbial deactivating levels of HPV. After a decontaminating QHPV loaded cycle, that HPV had degraded to undetectable <1 ppm levels inside the QHPV was confirmed using a handheld low-level hydrogen peroxide monitor (Drager Safety, Lubeck, Germany) that had been placed inside an IsoIVC-P prior to starting the QHPV loaded cycle. In addition, sterile swabs (FLOQ, Copan Flock Technologies, Brescia, Italy) were used to collect specimens from the exterior surfaces of the HPV-exposed IsoIVC-P and other surfaces inside the QHPV after completing a QHPV loaded cycle, by tracing a pattern as the swab tip was rolled, and then subjecting these specimens to both PCR for bacterial 16s rRNA and microbiological cultures, as described below.

Autoclave Sterilization: In the vivarium, cage wash movement is one-way, from soiled to clean to sterile. IsoIVC-P are sanitized and exposed to a 180° F. final rinse in an Atlantis cage and rack washer or an Arcadia tunnel washer, each with dryers (Tecniplast). Washed and dried caging equipment exit these machines and enter into a clean cage preparation room. Clean IsoIVC-P were stocked with feed, bedding and enrichments, reassembled, and clamped shut. Prepared IsoIVC-P and filled animal drinking water reservoir bottles were sterilized in separate loads by passage through 2 roll-in pre-vacuum model SNA-2000 bulk steam sterilizers with bio-seals, or a model SNA-1000, or a model SNA-500 autoclave (Matachana, Exton, PA). Appropriate autoclave processing was assured by using chemical and biologic indicators.

IsoIVC-P were sterilized as a unit held firmly closed on a sterilization transport rack (Tecniplast) unlatched, with white safety buttons up, and each supply air valve actuated open. Feed was extruded 5VOF-IRR 25 #Irradiated Purina Select Rodent Lab Diet 50 IF/6F Auto (Purina, St. Louis, MO). Bedding was paper cellulose Alpha Dri-IRR (Shepherd Specialty Paper, Watertown, TN). Enrichments included Nestlet (Ancare, Bellmore, NY) and Mouse Igloo (BioServ, Flemington, NJ). Assembled IsoIVC-P were sterilized using an autoclave cycle of 2.45 psia pre-vacuum, 121° C., 20 minutes, 2.45 psia drying vacuum, and validated as sterile using a Verify Steam Test Pack with Self Contained Biological Indicator and Integrator strip (Steris, Mentor OH) placed in the center “indicator” IsoIVC-P of the sterilization transport rack. IsoIVC-P cage level HEPA filters were discarded after 5 autoclave sterilization cycles and replaced.

Reverse osmosis-purified drinking water in Corning 1 L high temperature red-capped glass reservoir bottles was sterilized using an autoclave cycle of 122° C., 90 minutes, 2.45 psia vacuum (i.e., no pre- or post-pulse stages) with a MagnaAmp indicator (Steris) suspended inside a filled uncapped “indicator” bottle, and a dated Integrator strip (Steris) taped to the Tyvek cover of the water reservoir bottle basket load.

Animals: All mice were housed, cared for, and used in accordance with IACUC approved protocols. Male and female germfree mice each of the C57BL/6, BALB/c, and Swiss Webster lines were imported (Taconic, Germantown, NY) and separately mated to establish colony production. Germfree mice were monitored on arrival and at a minimum during each IsoIVC-P change-out every third week for 74 weeks for evidence of microbes by bacterial 16s rRNA PCR and by microbial cultures, as described below. Altered Schaedler Flora (ASF) associated mice (Taconic) were also imported and used to establish an ASF-associated C57BL/6 colony. Feces from germfree mice, ASF-associated mice, or FMT gavage-administered mice were collected as expressed and evaluated by shotgun whole genomic sequencing for evidence of microbes, as described below.

Fecal Microbial Transplants: Murine fecal pellets were collected as expressed from SPF or ASF mice in microcentrifuge tubes and frozen at −20° C. Slurries were prepared using sterile supplies in a biosafety cabinet from ten frozen fecal pellets by homogenizing the pellets in 800 μl of phosphate buffered saline (PBS) in a 1.5 ml microcentrifuge tube using a tuberculin syringe plunger to disrupt the pellets. This suspension was then transferred into 10 ml of PBS in a 15 ml conical tube, further homogenized, then strained through a 100 μm filter placed over a 50 ml conical tube. This filtrate was used to create 1.0 ml aliquots that were frozen at −80° C. until 100 μl fecal microbial transplant (FMT) gavage administrations to germfree mice. Approximately 200 μl of filtrate were submitted for microbial characterization, as described below.

QHPV Environmental Monitoring: Conditions inside the QHPV during processing mode involving germfree and gnotobiotic murine procedures were evaluated by testing the air circulating in the QHPV for airborne particles and for the presence of live airborne microbes. A calibrated Lighthouse Solair 3100 (Lighthouse Worldwide Solutions, Fremont, CA) light-scattering airborne particle counter was used to document airborne particles during 2-minute air sampling intervals. Airborne particles were quantified as the total number of particles of sizes ≥0.5 μm or ≥5 μm in 1 m³ air circulating within the QHPV. Also, a calibrated Active Air Sampler 4015 (Pharmagraph, Wokingham, UK), which draws 180 liters of QHPV air per minute for 5 minutes onto the contact surface of a 90 mm environmental culture plate containing trypticase soy agar plus 5% sheep blood (Thermo Fisher Scientific, Waltham MA), was used to assess for the presence of live airborne microbes during processing mode involving germfree and gnotobiotic murine procedures. QHPV air was tested for airborne particles and live microbes during gavage administrations and during germfree and gnotobiotic cage changeouts.

Microbial PCR Molecular Analysis: Total nucleic acids were extracted from specimens, including swabs, slurries, or fecal pellets using a commercially available platform (NucleoMag® VET Kit, Macherey-Nagel Bethlehem, PA). The hydrolysis probe-based Generic Bacteria 16S rRNA Gene real-time PCR assay used was based on the IDEXX BioAnalytics proprietary service platform (IDEXX Laboratories, Westbrook, ME). Real-time PCR analysis was performed at IDEXX BioAnalytics (Columbia, MO) using standard primer and probe concentrations (Applied BioSystems™), a commercially available master mix LightCycler® 480 Probes Master (Roche Applied Science, Indianapolis, IN), and a commercially available instrument (LightCycler® 480, Roche Applied Science). In addition to assay-specific positive and negative controls, an additional multiplexed fluorogenic real-time PCR assay targeting a mammalian gene (18s rRNA) and a universal bacterial reference gene (16s rRNA) was also amplified for each sample to ensure amplifiable nucleic acid recovery and to confirm the absence of PCR inhibition.

Microbial Culture & Identification: Microbial cultures were performed at the microbiology laboratory of IDEXX BioAnalytics. Sterile PBS was added to fecal pellet specimens to make a slurry in order to inoculate a proprietary battery of eight permissive culture media that were incubated aerobically and one permissive culture medium that was incubated anaerobically. Microbial colony types were harvested for proteomic analysis using a previously described direct transfer method (46). Harvested microbial colonies were overlaid with 1 μL of a saturated matrix solution of α-cyano-4-hydroxycinnamic acid in 50% acetonitrile and 2.5% trifluoroacetic acid (HCCA, Bruker Daltronics, Billerica, MA) on an etched steel target. The matrix solution was allowed to dry, and microbial colonies were analyzed by matrix-assisted laser desorption/ionization time-of-flight mass spectrometry (MALDI-TOF MS) using a mass spectrometer (Microflex™, Bruker Daltronics) and flexControl™ software (Bruker Daltronics). The organisms were identified using automated analysis by MALDI BioTyper® software (Bruker Daltronics) which compares the collected spectra with an integrated reference spectral database.

Whole-genome shotgun sequencing: Two fecal pellets from each mouse were collected freshly expressed into an individual, barcoded sample collection tube (Transnetyx; Cordova, TN) containing DNA stabilization buffer to ensure reproducibility, stability, and traceability, and shipped for DNA extraction, library preparation, and sequencing by Transnetyx. DNA extraction was optimized and fully automated using a Qiagen DNeasy 96 PowerSoil Pro QIAcube HT extraction kit. After DNA extraction, genomic DNA was converted into sequencing libraries using the KAPA HyperPlus library preparation protocol, a method optimized for minimal bias. Unique dual indexed adapters were used to ensure that reads and/or organisms were not mis-assigned. Libraries were sequenced using the Illumina NovaSeq instrument and protocol, a shotgun sequencing method at a depth of 2 million 2×150 bp read pairs, which enables species and strain level taxonomic resolution. Raw sequencing data in the form of FASTQ files were uploaded onto and analyzed using the One Codex database consisting of >115K whole microbial reference genomes. Comparing a microbial sample against the One Codex database consists of three sequential steps. First, every individual sequence is compared against the One Codex database by exact alignment using k-mers where k=31 (47). Second, based on the relative frequency of unique k-mers in the sample, sequencing artifacts are filtered out of the sample. Third, the relative abundance of each microbial species is estimated based on the depth and coverage of sequencing across every available reference genome. Classification results were filtered through several statistical post-processing steps designed to eliminate false positive results caused by contamination or sequencing artifacts, screen-out human and mouse reads, and remove low-quality reads.

Microscopy & Anesthesia: An Emspira-3 (Leica Microsystems, Wetzlar, Germany) was used for microsurgical procedures, and Pall MGF in-line gas filters (Pall Medical, Port Washington, NY) were used on induction box and rebreathing rodent facemask anesthesia lines leading from an isoflurane vaporizer, staged outside of the QUBE, into the QHPV via a tri-clover port adapter.

Statistical analysis: Differences in the alpha diversity parameters and relative abundance of taxonomic units was determined by using the Wilcoxon method for nonparametric comparisons for all pairs (version 16, JMP). P-values were adjusted for multiple comparisons according to the Benjamini-Hochberg false discovery rate with an adjusted alpha set to 0.05. Composition visualization was achieved using JMP version 16.

### Example 2 References

1. Foster K R, Schluter J, Coyte K Z, Rakoff-Nahoum S. The evolution of the host microbiome as an ecosystem on a leash. Nature 548:43-51 (2017).
2. Simon J C, Marchesi J R, Mougel C, Selosse M A. Host-microbiota interactions: from holobiont theory to analysis. Microbiome 7:5 (2019).
3. Bridges A A, Prentice J A, Wingreen N S, Bassler B L. Signal transduction network principles underlying bacterial collective behaviors. Annu Rev Microbiol 76:235-257 (2022).
4. Thaiss C A, Zmora N, Levy M, Elinav E. The microbiome and innate immunity. Nature 535:65-74 (2016).
5. Turnbaugh P J, Ley R E, Hamady M, Fraser-Liggett C M, Knight R, Gordon J I. The human microbiome project. Nature 449:804-810 (2007).
6. Alexander M, Turnbaugh P J. Deconstructing mechanisms of diet-microbiome-immune interactions. Immunity 53:264-276 (2020).
7. Macpherson A J, McCoy K D. Standardized animal models of host microbial mutualism. Mucosal Immunol 8 (3): 476-486 (2015).
8. Eberl G. Addressing the experimental variability associated with the microbiota. Mucosal Immunol 8 (3): 487-490 (2015).
9. Lam, K N, Alexander M, Turnbaugh P J. Precision medicine goes microscopic:
   engineering the microbiome to improve drug outcomes. Cell Host & Microbe 2622-34 (2019).
10. Faith J J, Rey F E, O'Donnell D, Karlsson M, McNulty N P, Kallstrom G, Goodman A L, Gordon J I. Creating and characterizing communities of human gut microbes in gnotobiotic mice. ISME J 4:1094-1098 (2010).
11. Routy B, et al. Gut microbiome modulates response to anti-PD-1 immunotherapy in melanoma patients. Science 359 (6371): 97-103 (2018).
12. Gopalakrishnan V, Spencer C N, Nezi L, Reuben A, Andrews M C, Karpinets T V, Prieto P A, Vicente D, Hoffman K, Wei S C, et al. Gut microbiome modulates response to anti-PD-1 immunotherapy in melanoma patients. Science 359:97-103 (2018).
13. Matson V, Fessler J, Bao R, Chongsuwat T, Zha Y, Alegre M L, Luke J J, Gajewski T F. The commensal microbiome is associated with anti-PD-1 efficacy in metastatic melanoma patients. Science 359:104-108 (2018).
14. Routy B, Chatelier E, Derosa L, Duong C, Alou M T, Daillère R, Fluckiger A, Messaoudene M, Rauber C, Roberti M P, et al. Gut microbiome influences efficacy of PD-1-based immunotherapy against epithelial tumors. Science 359:91-97 (2018).
15. Ridaura V K, Faith J J, Rey F E, Cheng J, Duncan A E, Kau A L, Griffin N W, Lombard V, Henrissat B, Bain J R, et al. Gut Microbiota from Twins Discordant for Obesity Modulate Metabolism in Mice. Science 341:1241214 (2013).
16. Buffie C G, Bucci V, Stein R R, McKenney P T, Ling L, Gobourne A, No D, Liu H, Kinnebrew M, Viale A, Littmann E, et al. Precision microbiome reconstitution restores bile acid mediated resistance to Clostridium difficile. Nature 517:205-208 (2015).
17. Buffington S A, Dooling S W, Sgritta M, Noecker C, Murillo O D, Felice D F, Turnbaugh P J, Costa-Mattioli M. Dissecting the contribution of host genetics and the microbiome in complex behaviors. Cell 184:1740-1756.e16 (2021).
18. Sharon G, Cruz N J, Kang D W, Gandal M J, Wang B, Kim Y M, Zink E M, Casy C P, Taylor B C, Lane C J, et al. Human gut microbiota from autism spectrum disorder promote behavioral symptoms in mice. Cell 177:1600-1618.e17 (2019).
19. van der Lelie D, Oka A, Taghavi S, Umeno J, Fan T J, Merrell K E, Watson S D, Ouellette L, Liu B, Awoniyi, et al. Rationally designed bacterial consortia to treat chronic immune-mediated colitis and restore intestinal homeostasis. Nat Commun 12:3105 (2021).
20. Schoeb T R, Rahija R J, Boyd C, Orcutt R P, Eaton K A. Principles of Establishing and Operating a Gnotobiotic Facility. Pg 22-58. In Gnotobiotics, T R Schoeb, K A Eaton, eds, Academic Press, Elsevier, London, U K (2017).
21. Boyd C, Eaton K A, Schoeb T R, Swennes A G, Bickerton H, Parsons B J, Sinclair S, Vowles C. Procedures for Rodents. Pg 66-333. In Gnotobiotics, T R Schoeb, K A Eaton, eds, Academic Press, Elsevier, London, U K (2017).
22. Fontaine C A, Skorupski A M, Vowles C J, Anderson N E, Poe S A, Eaton K A. How free of germs is germ-free? Detection of bacterial contamination in a germ-free mouse unit. Gut Microbes 6:4, 225-233 (2015).
23. Smith K, McCoy K D, Macpherson A J. Use of axenic animals in studying the adaptation of mammals to their commensal intestinal microbiota. Sem. Immunol. 19:59-69 (2007).
24. Ka Y, Ogura T, Tomiyama K, Ueno M, Nozu R, Tsuruzono N, Nozawa Y, Hamano M, Takakura A, Takahasi R. Creation of an experimental rearing environment for microbiome animal research using an individually ventilated cage system and biobubble enclosure. Exp. Anim. 70:177-184 (2021).
25. Rodriguez-Palacios A, Aladyshkina N, Ezeji J C, Erkkila H L, Conger M, Ward J, Webster J, Cominelli F. Cyclical bias in microbiome research revealed by a portable germfree housing system using nested isolation. Sci Reports 8:3801 (2018).
26. Basic M, Bolsega S, Smoczek A, Glasner J, Hiergeist A, Eberl C, Stecher B, Gessner A, Bleich A. Monitoring and contamination incidence of gnotobiotic experiments performed in microisolator cages. Int J Med Microbiol 311:151482 (2021).
27. Hecht G, Bar-Nathan C, Milite G, Alon I, Moshe Y, Greenfeld L, Dotsenko N, Suez J, Levy M, Thaiss C A, Dafni H, Elinav E, Harmelin A. A simple cage-autonomous method for the maintenance of the barrier status of germ-free mice during experimentation. Lab Anim. October 48 (4): 292-297 (2014).
28. Paik J, Pershutkina O, Meeker S, Yi J J, Dowling S, Hsu C, Hajjar A M, Maggio-Price L, Beck DAC. Potential for using a hermetically-sealed, positive-pressured isocage system for studies involving germ-free mice outside a flexible-film isolator. Gut Microbes July 4; 6 (4): 255-65 (2015).
29. Stehr M, Grewwling M C, Tischer S, Singh M, Blocker H, Monner D A, Muller W. Charles River altered Schaedler flora (CRASF) remained stable for four years in a mouse colony housed in individually ventilated cages. Lab Anim. 43:362-370 (2009).
30. M Brielmeier, E Mahabir, J R Needham, C Lengger, P Wilhelm, J Schmidt. Microbiological monitoring of laboratory mice and biocontainment in individually ventilated cages: a field study. Lab Anim July; 40 (3): 247-60 (2006).
31. Hufeldt M R, Nielsen D S, Vogensen F K, Midtvedt T, Hansen A K. Variation in the gut microbiota of laboratory mice is related to both genetic and environmental factors. Comp Med October; 60 (5): 336-47 (2010).
32. Thoene-Reineke C, Fischer A, Friese C, Briesemeister D, Göbel U B, Kammertoens T, Bereswill S, Heimesaat M M. Composition of intestinal microbiota in immune-deficient mice kept in three different housing conditions. PLOS One November 17; 9 (11): e113406 (2014).
33. Lundberg R, Bahl M I, Licht T R, Toft M F, Hansen A K. Microbiota composition of simultaneously colonized mice housed under either a gnotobiotic isolator or individually ventilated cage regime. Sci Rep 7:42245 (2017).
34. Ragland N H, Miedel E L, Gomez J M, Engelman R W. Staphylococcus xylosus PCR-validated Decontamination of Murine Individually Ventilated Cage Racks and Air Handling Units by Using ‘Active-Closed’ Exposure to Vaporized Hydrogen Peroxide. JAALAS 56 (6): 742-751 (2017).
35. Miedel E L, Ragland N H, Engelman R W. Facility wide eradication of Corynebacterium bovis using PCR validated vaporized hydrogen peroxide. JAALAS 57:465-476 (2018).
36. Ragland N H, Miedel E L, Engelman R W. PCR Prevalence of Murine Opportunistic Microbes and their Mitigation by Using Vaporized Hydrogen Peroxide. JAALAS 58 (2): 208-215 (2019).
37. Vedder A R, Miedel E L, Ragland N H, Balasis M E, Letson C T, Engelman R W, Padron E. Effects of Corynebacterium bovis on Engraftment of Patient-derived Chronic-Myelomonocytic Leukemia Cells in NSGS Mice. Comp Med 69 (4): 276-282 (2019).
38. Miedel E L, Ragland N H, Slate A R, Engelman R W. Persistent Corynebacterium bovis Infectious Hyperkeratotic Dermatitis in Immunocompetent Epidermal-Mutant dep/dep Mice. Vet Pathol 57 (4): 586-589 (2020).
39. Lemmen S, Scheithauer S, Hafner H, Yezli S, Mohr M, Otter J A. Evaluation of hydrogen peroxide vapor for the inactivation of nosocomial pathogens on porous and nonporous surfaces. Am J Infect Control 43:82-85 (2015).
40. Otter J A, Mepham S, Athan B, Mack D, Smith R, Jacobs M, Hopkins S. Terminal decontamination of the Royal Free London's high-level isolation unit after a case of Ebola virus disease using hydrogen peroxide vapor. Am J Infect Control 44:233-235 (2016).
41. Otter J A, Yezli S, Schouten M A, van Zanten A R, Houmes-Zielman G, Nohlmans-Paulssen M. Hydrogen peroxide vapor decontamination of an intensive care unit to remove environmental reservoirs of multidrug-resistant gram-negative rods during an outbreak. Am J Infect Control 38:754-756 (2010).
42. Otter J A, Cummins M, Ahmad F, van Tonder C, Drabu Y J. Assessing the biological efficacy and rate of recontamination following hydrogen peroxide vapor decontamination. J Hosp Infect 67:182-188 (2007).
43. Bier M E. 1987. Method of vaporizing multicomponent liquids. U.S. Pat. No. R E. 4,642,165 [Cited 15 Jun. 2017]. Available at: http://patft.uspto.gov/netacgi/nphParser?Sect1=PTO1&Sect2=HITOFF&d=PALL&p=1&u=/netah tml/PTO/srchnum.htm&r=1&f=G&I=50&s1=4642165.PN.&OS=PN/4642165&RS=PN/4642165
44. Grignol G, Eddington D, Karle D, Rickloff J. 2000. Chemical and biological aspects of hydrogen peroxide gas, Proceedings of the ISPE Barrier Isolation Technology Conference. Arlington, Virginia, 5-6 Jun. 2000. [Cited 15 Jun. 2017]. Available at: http://www.isolationinfo.com/pubs.asp
45. Hultman C, Hill A, McDonnell G. 2007. The physical chemistry of decontamination with gaseous hydrogen peroxide. Pharm Engin 27:22-32 (2007).
46. Philips B H, Crim M J, Hankenson F C, Steffen E K, Klein P S, Brice A K, Carty A J. Evaluation of presurgical skin preparation agents in African clawed frogs (Xenopus laevis). JAALAS 54:788-798 (2015).
47. Wood D E, Salzberg S L. Kraken: ultrafast metagenomic sequence classification using exact alignments. Genome Biol 15 (3): R46 (2014).
48. Weiner C M. Controlling the macroenvironment: A novel approach to germ free derivations. Taconic Biosciences' Webinar (2019).
49. Roesch P L. Microbiological monitoring of germ-free and gnotobiotic colonies. Taconic Biosciences' Webinar (2020).

### Aspects of the Disclosure

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A system for performing concurrent animal studies in the same room, comprising one or more isolation individually ventilated cages with HEPA-filtered positive-pressurization (IsoIVC-P), wherein each of the cages houses at least one animal that is germfree, a fecal transplantee, or gnotobiotic; one or more support racks comprising an air handling unit, wherein the air handling unit provides filtered air to each of the IsoIVC-Ps when the one or more IsoIVC-Ps are stored on the support rack, wherein each of the one or more IsoIVC-Ps is self-hermetically sealed when removed from the support rack; and one or more isolators, where each isolator is an aseptic glovebox isolator comprising a hydrogen peroxide vapor module.

Aspect 2. The system of aspect 1, further comprising an anesthesia administration system comprising: an anesthesia line connected to the isolator chamber floor via a port adapter, wherein the anesthesia line comprises a first breathing system filter disposed in the anesthesia line prior to an induction box of the isolator and a second breathing system filter disposed in the anesthesia line outside of the isolator after a vaporizer of the isolator, such that anesthesia is provided to the inside of the isolator and contaminants are prevented from entering the isolator.

Aspect 3. A method for performing concurrent animal studies in the same room, comprising: sterilizing, via autoclave, one or more isolation individually ventilated cages with HEPA-filtered positive-pressurization (IsoIVC-P); providing an animal to each of the one or more sterilized IsoIVC-Ps to form one or more animal-occupied IsoIVC-Ps; providing the one or more animal-occupied IsoIVC-Ps to an isolator, where the isolator is an aseptic glovebox isolator comprising a hydrogen peroxide vapor module and wherein the one or more animal-occupied IsoIVC-Ps are hermetically sealed while in the isolator; sterilizing, via hydrogen peroxide vapor, the one or more animal-occupied IsoIVC-Ps; and opening one of the one or more animal-occupied IsoIVC-Ps inside the isolator to perform a study.

Aspect 4. The method of aspect 3, further comprising a plurality of isolators, and wherein each of the one or more animal-occupied IsoIVC-Ps provided to a given isolator contains animals having an identical status to one another, wherein the status is germfree or gnotobiotic.

Aspect 5. The method of aspect 3 or aspect 4, wherein the animal is a mouse.

Aspect 6. The method of aspect 5, wherein the mouse is a germ-free mouse.

Aspect 7. The method of aspect 5, wherein the mouse is a gnotobiotic mouse.

Aspect 8. The method of aspect 3, wherein when the one or more animal-occupied IsoIVC-Ps are not in an isolator, the one or more animal-occupied IsoIVC-Ps are stored on supporting racks, wherein the supporting racks comprise an air-handling unit.

Aspect 8a. The method of aspect 8, wherein the mouse is a germ-free mouse.

Aspect 8b. The method of aspect 8, wherein the mouse is a gnotobiotic mouse.

Aspect 9. The method of any of aspects 3-8b, wherein when stored on the racks, a given one of the animal-occupied IsoIVC-Ps is occupied by an animal from a same or a different population than an animal occupying a neighboring animal-occupied IsoIVC-P.

Aspect 10. The method of any of aspects 3-9, wherein the IsoIVC-Ps comprise sterilized bedding, feed, and bottles, wherein sterilization of the bedding, feed, and bottles is performed separately from the sterilization of the one or more IsoIVC-Ps.

Aspect 11. The method of any of aspects 3-9, wherein the IsoIVC-Ps comprise sterilized bedding, feed, and bottles, wherein sterilization of the bedding, feed, and bottles is performed concurrently with the sterilization of the one or more IsoIVC-Ps.

Aspect 12. The method of any of aspects 3-11, wherein the providing step comprises providing up to 5 animal-occupied IsoIVC-Ps to the isolator for up to 2 hours without detrimental effect on the animals.

Aspect 13. The method of any of aspects 3-12, wherein the hydrogen peroxide vapor sterilization step lasts about 45 minutes (e.g., about 40 minutes to 50 minutes).

Aspect 14. The method of any of aspects 3-13, further comprising administering anesthesia to the isolator chamber floor via an anesthesia line connected to the isolator chamber floor, wherein the anesthesia line is connected to the isolator via a port adapter, wherein the anesthesia line comprises a first breathing system filter disposed in the anesthesia line prior to an induction box of the isolator and a second breathing system filter disposed in the anesthesia line outside of the isolator after a vaporizer of the isolator, such that anesthesia is provided to the inside of the isolator and contaminants are prevented from entering the isolator.

Aspect 15. The systems and methods according to any of the preceding aspects, wherein the animals are hermetically sealed within the IsoIVC-Ps for up to about 2 hours without detrimental effect to the animals, e.g., up to 50 minutes, up to 55 minutes, up to 60 minutes, up to 65 minutes, up to 70 minutes, up to 75 minutes, up to 80 minutes, up to 85 minutes, up to 90 minutes, up to 95 minutes, up to 100 minutes, up to 105 minutes, up to 110 minutes, up to 115 minutes, or up to 120 minutes.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

What is claimed is:

1. A system for performing concurrent animal studies in the same room, comprising:
one or more isolation individually ventilated cages with HEPA-filtered positive-pressurization (IsoIVC-P), wherein each of the cages houses at least one animal that is germfree, a fecal transplantee, or gnotobiotic;
one or more support racks comprising an air handling unit,
wherein the air handling unit provides filtered air to each of the IsoIVC-Ps when the one or more IsoIVC-Ps are stored on the support rack,
wherein each of the one or more IsoIVC-Ps is self-hermetically sealed when removed from the support rack; and
one or more isolators, where each isolator is an aseptic glovebox isolator comprising a hydrogen peroxide vapor module.

2. The system of claim 1, further comprising an anesthesia administration system comprising:
an anesthesia line connected to the isolator chamber floor via a port adapter, wherein the anesthesia line comprises a first breathing system filter disposed in the anesthesia line prior to an induction box of the isolator and a second breathing system filter disposed in the anesthesia line outside of the isolator after a vaporizer of the isolator, such that anesthesia is provided to the inside of the isolator and contaminants are prevented from entering the isolator.

3. A method for performing concurrent animal studies in the same room, comprising:
sterilizing, via autoclave, one or more isolation individually ventilated cages with HEPA-filtered positive-pressurization (IsoIVC-P);
providing an animal to each of the one or more sterilized IsoIVC-Ps to form one or more animal-occupied IsoIVC-Ps;
providing the one or more animal-occupied IsoIVC-Ps to an isolator, where the isolator is an aseptic glovebox isolator comprising a hydrogen peroxide vapor module and wherein the one or more animal-occupied IsoIVC-Ps are hermetically sealed while in the isolator;
sterilizing, via hydrogen peroxide vapor, the one or more animal-occupied IsoIVC-Ps; and
opening one of the one or more animal-occupied IsoIVC-Ps inside the isolator to perform a study.

4. The method of claim 3, further comprising a plurality of isolators, and wherein each of the one or more animal-occupied IsoIVC-Ps provided to a given isolator contains animals having an identical status to one another, wherein the status is germfree or gnotobiotic.

5. The method of claim 3, wherein the animal is a mouse.

6. The method of claim 5, wherein the mouse is a germ-free mouse.

7. The method of claim 5, wherein the mouse is a gnotobiotic mouse.

8. The method of claim 3, wherein when the one or more animal-occupied IsoIVC-Ps are not in an isolator, the one or more animal-occupied IsoIVC-Ps are stored on supporting racks, wherein the supporting racks comprise an air-handling unit.

9. The method of claim 8, wherein when stored on the racks, a given one of the animal-occupied IsoIVC-Ps is occupied by an animal from a same or a different population than an animal occupying a neighboring animal-occupied IsoIVC-P.

10. The method of claim 3, wherein the IsoIVC-Ps comprise sterilized bedding, feed, and bottles, wherein sterilization of the bedding, feed, and bottles is performed separately from the sterilization of the one or more IsoIVC-Ps.

11. The method of claim 3, wherein the IsoIVC-Ps comprise sterilized bedding, feed, and bottles, wherein sterilization of the bedding, feed, and bottles is performed concurrently with the sterilization of the one or more IsoIVC-Ps.

12. The method of claim 3, wherein the providing step comprises providing up to 5 animal-occupied IsoIVC-Ps to the isolator for up to 2 hours without detrimental effect on the animals.

13. The method of claim 12, wherein the hydrogen peroxide vapor sterilization step lasts about 45 minutes.

14. The method of claim 3, further comprising administering anesthesia to the isolator chamber floor via an anesthesia line connected to the isolator chamber floor, wherein the anesthesia line is connected to the isolator via a port adapter, wherein the anesthesia line comprises a first breathing system filter disposed in the anesthesia line prior to an induction box of the isolator and a second breathing system filter disposed in the anesthesia line outside of the isolator after a vaporizer of the isolator, such that anesthesia is provided to the inside of the isolator and contaminants are prevented from entering the isolator.
